SEQUENCE LISTING (13 sequences)

SEQ ID NO: 1 - DNA, 690 bp, Vesicular stomatitis Indiana virus
  SITE (1)..(690): VSV Indiana serotype
atgagttccttaaagaagattctcggtctgaaggggaaaggtaagaaatctaagaaattagggatcgcaccacccccttatgaagaggacactaacatggagtatgctccgagcgctccaattgacaaatcctattttggagttgacgagatggacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 2 - DNA, 690 bp, Artificial Sequence
  Synthetic Mutant G21E/L111A/M51R
atgagttccttaaagaagattctcggtctgaaggggaaaggtaagaaatctaagaaattagaaatcgcaccacccccttatgaagaggacactaacatggagtatgctccgagcgctccaattgacaaatcctattttggagttgacgagcgagacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttgcaggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 3 - protein, 229 aa, Vesicular stomatitis Indiana virus
  SITE (1)..(229): VSV Indiana serotype
MSSLKKILGLKGKGKKSKKLGIAPPPYEEDTNMEYAPSAPIDKSYFGVDEMDTHDPHQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 4 - protein, 229 aa, Artificial Sequence
  Synthetic Mutant G21E/L111A/M51R
MSSLKKILGLKGKGKKSKKLEIAPPPYEEDTNMEYAPSAPIDKSYFGVDERDTHDPHQLRYEKFFFTVKMTVRSNRPFRTYSDVAAAVSHWDHMYIGMAGKRPFYKILAFAGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 5 - DNA, 686 bp, Vesicular stomatitis New Jersey virus
  SITE (1)..(686): VSV New Jersey serotype Wild Type
atgagttccttcaaaaagattctgggattttcttcaaaaagtcacaagaaatcaaagaaactaggcttgccacctccttatgaggaatcaagtcctatggagattcaaccatctgccccattatcaaatgacttcttcggaatggaggatatggatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 6 - DNA, 686 bp, Artificial Sequence
  Synthetic G22E/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 7 - DNA, 686 bp, Artificial Sequence
  Synthetic G22E/L110A/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctgcaattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 8 - protein, 229 aa, Vesicular stomatitis New Jersey virus
  SITE (1)..(229): VSV New Jersey serotype Wild Type
MSSFKKILGFSSKSHKKSKKLGLPPPYEESSPMEIQPSAPLSNDFFGMEDMDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 9 - protein, 229 aa, Artificial Sequence
  Synthetic G22E/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 10 - protein, 229 aa, Artificial Sequence
  Synthetic G22E/L110A/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIAAIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 11 - protein, 21 aa, Apis mellifera
  SITE (1)..(21): Honeybee melittin signal peptide
MKFLVNVALVFMVVYISYIYA

SEQ ID NO: 12 - protein, 49 aa, Unknown
  Synthetic VSV G protein transmembrane domain and cytoplasmic tail
SSIASFFFIIGLIIGLFLVLRVGIYLCIKLKHTKKRQIYTDIEMNRLGK

SEQ ID NO: 13 - DNA, 25 bp, Unknown
  Synthetic VSV intergenic junctions
catatgaaaaaaactaacagatatc

(19) United States
(12) Patent Application Publication
(10) Pub. No.: US 2024/0139311 A1
(43) Pub. Date: May 2, 2024

(54) RECOMBINANT VSV-SARS-COV-2 VACCINE

(71) Applicant: Sumagen Canada Inc., London (CA)

(72) Inventors: Chil-Yong KANG, London (CA); Gyoung Nyoun KIM, London (CA); Kunyu WU, London (CA); Nasrin SAEEDIAN, London (CA)

(73) Assignee: Sumagen Canada Inc., London, ON (CA)

(21) Appl. No.: 18/280,147

(22) PCT Filed: Feb. 25, 2022
(86) PCT No.: PCT/CA2022/050277

§ 371 Date: Sep. 1, 2023

Related U.S. Application Data
(60) Provisional application No. 63/156,109, filed on Mar. 3, 2021.

(51) Int. Cl. A61K 39/215; A61P 31/14; A61P 37/04

(52) U.S. Cl. CPC A61K 39/215; A61P 31/14; A61P 37/04; A61K 2039/5256

(57) ABSTRACT

A recombinant vesicular stomatitis vims (rVSV) carrying one or more genes that encode for the spike protein of SARS-CoV-2 or for both the S protein and the envelope protein of the SARS-CoV-2. Vaccines, regimens and kits having the rVSV are used for the prevention of infections caused by SARS-CoV-2.

### FIELD OF THE INVENTION

The present invention relates to recombinant VSV-SARS-CoV-2, in particular to recombinant vesicular stomatitis viruses containing full or partial spike proteins and/or the envelope protein of the SARS-CoV-2, vaccines and prime-boost vaccines or immunogenic compositions against SARS-CoV-2.

### BACKGROUND OF THE INVENTION

Throughout this application, various references are cited in brackets to describe more fully the state of the art to which this invention pertains. The disclosure of these references is hereby incorporated by reference into the present disclosure.

In December 2019, a pneumonia associated with the 2019 novel SARS-CoV-2 emerged in Wuhan, China. As of Feb. 24, 2021, there are 112,516,000 reported COVID-19 cases worldwide, and 2,492,000 deaths. Cases are expected to increase continuously, and a third wave of infection is expected worldwide. The SARS-CoV-2 RNA genome sequence is known (1). SARS-CoV-2 is 96% identical at the whole-genome level to a bat coronavirus (RaTG13) indicating recent emergence and introduction into humans (2). SARS-CoV-2 is more distantly related to SARS-CoV (˜79%) and SARS-CoV-2 (˜50%). Evidence shows that SARS-CoV-2 can bind to the human angiotensin-converting enzyme 2 (hACE2) receptor, allowing for human-human transmission. Moreover, evidence suggests that the SARS-CoV-2 Spike protein binds hACE2 with higher affinity than SARS-CoV Spike protein, potentially contributing to a higher transmission rate (3). The evolution, adaptation, and spread of SARS-CoV-2 and future emerging coronaviruses warrant urgent investigation.

An ideal SARS-CoV-2 vaccine should induce completely protective immune responses, must be safe, relatively easy to administrate, and efficient for manufacturing.

The Applicant has developed a system comprising a combination of vaccines that elicits an immune response against SARS-CoV-2.

### SUMMARY OF THE INVENTION

In one embodiment, the present disclosure provides for a recombinant vesicular stomatitis virus (rVSV) carrying one or more genes that encode for (i) a spike (S) protein of SARS-CoV-2, or (ii) an envelope (E) protein of the SARS-CoV-2 or (iii) both the S protein and the E protein. In one aspect of the embodiment, the SARS-CoV-2 includes SARS-CoV-2 variants.

In one embodiment of the rVSV of the present disclosure, the S protein is a full length (S_{F}) or a partial length S protein of the SARS-CoV-2, and wherein the partial length S protein is one or more of a S1 subunit of the S_{F} protein, a S2 subunit of the S_{F} protein, or a receptor binding domain (RBD) of the S_{F} protein.

In another embodiment of the rVSV of the present disclosure, at least one of the S protein and the E protein include one or more modifications.

In another embodiment of the rVSV of the present disclosure, the one or more genes encode for both the S protein and the E protein, the S protein is the RBD, and wherein the one or more modifications are a honeybee melittin signal peptide (msp) at the NH₂-terminus of the RBD (msp-RBD), a VSV G protein transmembrane domain and cytoplasmic tail (Gtc) at the C-terminus of the RBD and a Gtc at the C-terminus of the E protein (rVSV-msp-RBD-Gtc+E-Gtc).

In another embodiment of the rVSV of the present disclosure, the one or more genes encode for both the S protein and the E protein, only the S protein includes the one or more modifications, the S protein is the RBD, and wherein the one or more modifications are a honeybee melittin signal peptide (msp) at the NH₂-terminus of the RBD (mspRBD) and a VSV G protein transmembrane domain and cytoplasmic tail (Gtc) at the C-terminus of the RBD (rVSV-msp-RBD-Gtc+E).

In another embodiment of the rVSV of the present disclosure, the one or more genes encode for the S protein, the S protein is the S_{F} protein, and wherein the one or more modifications are a signal peptide sequence of wild type S_{F} protein replaced with a honeybee melittin peptide (msp) at the NH₂-terminus of the S_{F} protein and a VSV G protein transmembrane domain and cytoplasmic tail (Gtc) at the C-terminus of the S_{F} protein (rVSV-msp-S_{F}-Gtc).

In another embodiment of the rVSV of the present disclosure, the one or more genes encode for the S protein and the S protein is the S_{F} (rVSV-S_{F}).

In another embodiment of the rVSV of the present disclosure, the one or more genes encode for the S protein, the S protein is the S1 protein, and wherein the one or more modifications are a signal peptide sequence of wild type S1 protein replaced with a honeybee melittin peptide (msp) at the NH₂-terminus of the S1 protein and a VSV G protein transmembrane domain and cytoplasmic tail (Gtc) at the C-terminus of the S1 protein (rVSV-msp-S1-Gtc).

In another embodiment of the rVSV of the present disclosure, the one or more genes encode for the S protein and the S protein is the S1 protein (rVSV-S1).

In another embodiment of the rVSV of the present disclosure, the Gtc is a VSV_{Ind} Gtc or a VSV_{NJ} Gtc.

In another embodiment of the rVSV of the present disclosure, the rVSV is a replication competent rVSV of Indiana (VSV_{Ind}) serotype.

In another embodiment of the rVSV of the present disclosure, the rVSV is a replication competent rVSV of New Jersey (VSV_{NJ}) serotype.

In another embodiment of the rVSV of the present disclosure, the VSV_{NJ} is Hazelhurst strain (VSV_{NJ-H}) or Ogden strain (VSV_{NJ-O}).

In another embodiment of the rVSV of the present disclosure, the rVSV_{Ind} includes a mutant matrix protein (M) gene.

In another embodiment of the rVSV of the present disclosure, the rVSV_{NJ} includes a mutant matrix protein (M) gene.

In another embodiment of the rVSV of the present disclosure, the mutant rVSV_{Ind} M protein includes a GML mutation (rVSV_{Ind}-GML).

In another embodiment of the rVSV of the present disclosure, the rVSV_{NJ} M protein includes a GMM mutation (rVSV_{NJ}-GMM) or a GMML mutation (rVSV_{NJ}-GMML).

In another embodiment of the rVSV of the present disclosure, the one or more genes are codon-optimized for expression in a human cell.

In another embodiment of the rVSV of the present disclosure, the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

In another embodiment, the present disclosure provides for a vaccine or immunological composition including a recombinant vesicular stomatitis virus (rVSV) according to an embodiment of the present disclosure.

In another embodiment, the present disclosure provides for a vaccine or immunological composition comprising the rVSV-msp-RBD-Gtc+E-Gtc.

In another embodiment, the present disclosure provides for a vaccine or immunological composition comprising the rVSV-msp-RBD-Gtc+E.

In another embodiment, the present disclosure provides for a vaccine or immunological composition comprising the rVSV-msp-S_{F}-Gtc.

In another embodiment, the present disclosure provides for a vaccine or immunological composition comprising the rVSV-S_{F}.

In another embodiment, the present disclosure provides for a vaccine or immunological composition comprising the rVSV-msp-S1-Gtc.

In another embodiment, the present disclosure provides for a vaccine or immunological composition comprising the rVSV-S1.

In another embodiment, the present disclosure provides for a prime boost immunization combination against SARS-CoV-2 including: (a) a prime vaccine or immunogenic composition comprising a replication competent recombinant vesicular stomatitis virus (rVSV) carrying one or more genes that encode for (i) a spike (S) protein of a SARS-CoV-2, or (ii) an envelope (E) protein of the SARS-CoV-2, or (iii) both the S protein and the E protein, and (b) a booster vaccine or immunogenic composition comprising a replication competent rVSV carrying the same one or more genes. In aspects of the embodiment, the SARS-CoV-2 includes SARS-CoV-2 variants.

In one embodiment of the prime boost immunization combination against SARS-CoV-2 of the present disclosure, the S protein is full length (S_{F}) or partial length, and wherein the partial length S protein is one or more of a S1 subunit of the S_{F} protein, a S2 subunit of the S_{F} protein, or a receptor binding domain (RBD) of the S_{F} protein.

In another embodiment of the prime boost immunization combination against SARS-CoV-2 of the present disclosure, the one or more genes encode for both the S protein and the E protein, the S protein is the RBD of the S protein, and wherein the RBD includes a honeybee melittin signal peptide (msp) at the NH₂-terminus of the RBD (msp-RBD) and a VSV G protein transmembrane domain and cytoplasmic tail (Gtc) at the C-terminus of the RBD and the E protein includes a Gtc at the C-terminus of the E protein (rVSV-msp-RBD-Gtc+E-Gtc).

In another embodiment of the prime boost immunization combination against SARS-CoV-2 of the present disclosure, the one or more genes encode for both the S protein and the E protein, the S protein is the RBD of the S protein, and wherein the RBD includes a honeybee melittin signal peptide (msp) at the NH₂-terminus of the RBD (msp-RBD) and a VSV G protein transmembrane domain and cytoplasmic tail (Gtc) at the C-terminus of the RBD (rVSV-msp-RBD-Gtc+E).

In another embodiment of the prime boost immunization combination against SARS-CoV-2 of the present disclosure, the one or more genes encode for the S protein, the S protein is the S_{F} protein having a honeybee melittin peptide (msp) at the NH₂-terminus of the S protein and a VSV G protein transmembrane domain and cytoplasmic tail (Gtc) at the C-terminus of the S protein (rVSV-msp-S_{F}-Gtc).

In another embodiment of the prime boost immunization combination against SARS-CoV-2 of the present disclosure, the one or more genes encode for the S protein, and the S protein is the S_{F} protein (rVSV-S_{F}).

In another embodiment of the prime boost immunization combination against SARS-CoV-2 of the present disclosure, the one or more genes encode for the S protein, the S protein is the S1 protein having a honeybee melittin peptide (msp) at the NH₂-terminus of the S1 protein and a VSV G protein transmembrane domain and cytoplasmic tail (Gtc) at the C-terminus of the S1 protein (rVSV-msp-S1-Gtc).

In another embodiment of the prime boost immunization combination against SARS-CoV-2 of the present disclosure, the one or more genes encode for the S protein and the S protein is the S1 protein (rVSV-S1).

In another embodiment of the prime boost immunization combination against SARS-CoV-2 of the present disclosure, the rVSV of the prime vaccine or immunogenic composition and the rVSV of the booster vaccine or immunogenic composition are rVSV of the same serotype.

In another embodiment of the prime boost immunization combination against SARS-CoV-2 of the present disclosure, the rVSV of the prime vaccine or immunogenic composition and the rVSV of the booster vaccine or immunogenic composition are rVSV of Indiana serotype (rVSV_{Ind}).

In another embodiment of the prime boost immunization combination against SARS-CoV-2 of the present disclosure, the rVSV of the prime vaccine or immunogenic composition and the rVSV of the booster vaccine or immunogenic composition are rVSV of New Jersey serotype (rVSV_{NJ}).

In another embodiment of the prime boost immunization combination against SARS-CoV-2 of the present disclosure, the rVSV of the prime vaccine or immunogenic composition is Indiana serotype (VSV_{Ind}) and the rVSV of the booster vaccine or immunogenic composition is New Jersey (VSV_{NJ}).

In another embodiment of the prime boost immunization combination against SARS-CoV-2 of the present disclosure, the rVSV of the prime vaccine or immunogenic composition is New Jersey serotype (rVSV_{NJ}) and the rVSV of the booster vaccine or immunogenic composition is rVSV of Indiana serotype (rVSV_{Ind}).

In another embodiment of the prime boost immunization combination against SARS-CoV-2 of the present disclosure, the rVSV of the prime vaccine and the rVSV of the booster vaccine include a mutant matrix protein (M) gene.

In another embodiment of the prime boost immunization combination against SARS-CoV-2 of the present disclosure, when the rVSV is rVSV_{Ind}, the M protein includes a GML mutation (rVSV_{Ind}-GML).

In another embodiment of the prime boost immunization combination against SARS-CoV-2 of the present disclosure, when the rVSV is rVSV_{NJ}, the M protein includes a GMM mutation-(rVSV_{NJ}-GMM) or a GMML mutation (rVSV_{NJ}-GMML).

In another embodiment of the prime boost immunization combination against SARS-CoV-2 of the present disclosure, the one or more genes are codon optimized for expression in a human cell.

In another embodiment of the prime boost immunization combination against SARS-CoV-2 of the present disclosure, the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

In another embodiment, the present disclosure provides for a method for inducing an immune response in a mammal against SARS-CoV-2, comprising administering to the mammal an effective amount of a vaccine or immunogenic composition according to an embodiment of the present disclosure.

In another embodiment, the present disclosure provides for a method for inducing an immune response in a mammal against SARS-CoV-2, comprising administering to the mammal the prime boost immunization combination according to an embodiment of the present disclosure.

In another embodiment, the present disclosure provides for a method for preventing an infection caused by a SARS-CoV-2 comprising administering to the mammal an effective amount of a vaccine or immunogenic composition according to an embodiment of the present disclosure.

In another embodiment, the present disclosure provides for a method for preventing an infection caused by a SARS-CoV-2 comprising administering to the mammal the prime boost immunization combination according to an embodiment of the present disclosure.

In another embodiment, the present disclosure provides for a use of a vaccine or immunogenic composition according to an embodiment of the present disclosure for the prevention or treatment of a SARS-CoV-2 infection.

In another embodiment, the present disclosure provides for a use of the prime boost immunization combination according to an embodiment of the present disclosure for the prevention or treatment of a SARS-CoV-2 infection.

In another embodiment, the present disclosure provides for a use of a rVSV of the present disclosure in the manufacture of a vaccine or a prime boost immunization combination for the prevention or treatment of a SARS-CoV-2 infection.

In another embodiment, the present disclosure provides for a kit comprising (a) at least one dose of an effective amount of a prime vaccine or immunogenic composition including a recombinant vesicular stomatitis virus (rVSV) carrying one or more genes that encode for (i) a spike (S) protein of a SARS-CoV-2, or (ii) an envelope (E) protein of the SARS-CoV-2 or (iii) both the S protein and the E protein, and (b) at least one dose of an effective amount of a booster vaccine or immunogenic composition comprising a rVSV carrying the same one or more genes. In one aspect of the embodiment, the SARS-CoV-2 includes SARS-CoV-2 variants.

In one embodiment of the kit of the present disclosure, the at least one dose of the prime vaccine or immunogenic composition and the at least one dose of the booster vaccine or immunological composition are formulated in a pharmaceutically acceptable carrier.

In another embodiment of the kit of the present disclosure, the rVSV of the prime vaccine or immunogenic composition and the rVSV of the booster vaccine or immunogenic composition are of Indiana serotype (rVSV_{Ind}).

In another embodiment of the kit of the present disclosure, the rVSV of the prime vaccine or immunogenic composition and the rVSV of the booster vaccine or immunogenic composition of claim 49 are of New Jersey serotype (rVSV_{NJ}).

In another embodiment of the kit of the present disclosure, the rVSV of the prime vaccine or immunological composition is Indiana (VSV_{Ind}) and the rVSV of the booster vaccine or immunological composition is of New Jersey serotype (VSV_{NJ}).

In another embodiment of the kit of the present disclosure, the rVSV of the prime vaccine or immunological composition is of New Jersey serotype (rVSV_{NJ}), and the rVSV of the booster vaccine or immunological vaccine is of Indiana serotype (rVSV_{Ind}).

In another embodiment of the kit of the present disclosure, the rVSV of the prime vaccine or immunological composition and the rVSV of the booster vaccine or immunological composition include a mutant matrix protein (M) gene.

In another embodiment of the kit of the present disclosure, when the rVSV is rVSV_{Ind}, the M protein includes a GML mutation (rVSV_{Ind}-GML), and when the rVSV is rVSV_{NJ} M protein includes a GMM mutation (rVSV_{NJ}-GMM) or a GMML mutation (rVSV_{NJ}-GMML).

In another embodiment of the kit of the present disclosure, the S protein is full length (S_{F}) or partial length, and wherein the partial length S protein is one or more of a S1 subunit of the S_{F} protein, a S2 subunit of the S_{F} protein, or a receptor binding domain (RBD) of the S_{F}.

In another embodiment of the kit of the present disclosure, the one or more genes encode for both the S protein and the E protein, the S protein is the RBD of the S_{F} protein, and wherein the RBD includes a honeybee melittin signal peptide (msp) at the NH₂-terminus of the RBD (msp-RBD) and a VSV G protein transmembrane domain and cytoplasmic tail (Gtc) at the C-terminus of the RBD and the E protein includes a Gtc at the C-terminus of the E protein (rVSV-msp-RBD-Gtc+E-Gtc).

In another embodiment of the kit of the present disclosure, the one or more genes encode for both the S protein and the E protein, the S protein is the RBD of the S_{F} protein, and wherein the RBD includes a honeybee melittin signal peptide (msp) at the NH₂-terminus of the RBD (msp-RBD) and a VSV G protein transmembrane domain and cytoplasmic tail (Gtc) at the C-terminus of the RBD (rVSV-msp-RBD-Gtc+E).

In another embodiment of the kit of the present disclosure, the one or more genes encode for the S protein, the S protein is the S_{F} protein having a honeybee melittin peptide (msp) at the NH₂-terminus of the S_{F} protein and a VSV G protein transmembrane domain and cytoplasmic tail (Gtc) at the C-terminus of the S_{F} protein (rVSV-msp-S_{F}-Gtc).

In another embodiment of the kit of the present disclosure, the one or more genes encode for the S protein and the S protein is the S_{F} (rVSV-S_{F}).

In another embodiment of the kit of the present disclosure, the one or more genes encode for the S protein, the S protein is the S1 protein having a honeybee melittin peptide (msp) at the NH₂-terminus of the S1 protein and a VSV G protein transmembrane domain and cytoplasmic tail (Gtc) at the C-terminus of the S1 protein (rVSV-msp-S1-Gtc).

In another embodiment of the kit of the present disclosure, the one or more genes encode for the S protein and the S protein is the S1 protein (rVSV-S1).

In another embodiment of the kit of the present disclosure, the kit further includes instructions to immunize a mammal against SARS-CoV-2.

In another embodiment of the kit of the present disclosure, the one or more genes are optimized for expression in a human cell.

In another embodiment of the kit of the present disclosure, the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

In another embodiment the present disclosure is a recombinant protein comprising an RBD of a S protein of SARS-CoV-2 and cytoplasmic tail (Gtc) at the C-terminus of the RBD and an E protein of SARS-CoV-2.

In another embodiment the present disclosure provides for a recombinant protein comprising an RBD of a S protein of SARS-CoV-2 and cytoplasmic tail (Gtc) at the C-terminus of the RBD and an E protein of SARS-CoV-2 having the Gtc at the C-terminus.

In another embodiment the present disclosure provides for a recombinant protein comprising a full-length S protein (S_{F}) of SARS-CoV-2 having a honeybee melittin peptide (msp) at the NH₂-terminus of the S protein and a VSV G protein transmembrane domain and cytoplasmic tail (Gtc) at the C-terminus of the S protein.

In another embodiment the present disclosure provides for a recombinant protein comprising a full-length S protein of SARS-CoV-2.

In another embodiment the present disclosure provides for a recombinant protein comprising S1 protein of SARS-CoV-2 having a honeybee melittin peptide (msp) at the NH₂-terminus of the S1 protein and a VSV G protein transmembrane domain and cytoplasmic tail (Gtc) at the C-terminus of the S1 protein.

In another embodiment the present disclosure provides for a recombinant protein comprising a S1 protein of SARS-CoV-2.

In another embodiment the present disclosure provides for a cell carrying a rVSV according to any embodiment of the present disclosure.

In another embodiment the present disclosure provides for a cell that secrets (i) a spike protein of SARS-CoV-2, or (ii) an envelope protein of SARS-CoV-2, or (iii) the spike protein of SARS-CoV-2 and the envelope protein of SARS-CoV-2.

In one embodiment of the cell of the present disclosure, the cell is an eukaryotic cell.

In another embodiment of the cell of the present disclosure, the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

### BRIEF DESCRIPTION OF THE DRAWINGS

The present disclosure will become more fully understood from the detailed description given herein and from the accompanying drawings, which are given by way of illustration only and do not limit the intended scope of the disclosure.

FIG. 1. Construction of recombinant rVSV_{Ind} and rVSV_{NJ} with S_{F} genes of SARS-CoV-2 with and without honeybee msp and VSV Gtc. Codon-optimized full-length Spike protein gene (S_{F}) of SARS-CoV-2 with and without 21 amino acids honeybee melittin signal peptide [(msp) SEQ ID NO: 11 NH₂-MKFLVNVALVFMVVYISYIYA-COOH] gene, and 49 amino acids VSV G protein transmembrane domain and cytoplasmic tail [(Gtc) SEQ ID NO: 12 NI-12-5 SIASFFFIIGLIIGLFLVLRVGIYLCIKLKHTKKRQIYTDIEMNRLGK-COOH] were inserted into the G and L gene junction of rVSV_{Ind} and rVSV_{NJ}. In addition, 25-nucleotides-long VSV intergenic junctions (SEQ ID NO: 13 5′-CATATGAAAAAAACTAACAGATATC-3′) were inserted between genes to provide transcription termination, polyadenylation and the transcription reinitiation sequences. Recombinant viruses were rescued by VSV reverse genetics.

pT7: Bacteriophage T7 promoter for DNA-dependent RNA polymerase. N: VSV Nucleocapsid Protein gene. P: VSV Phosphoprotein gene. M: VSV Matrix protein gene. G: VSV Glycoprotein gene. L: VSV Large protein, RNA-dependent RNA polymerase gene. l: Leader region in the 3′-end of the VSV genome. t: Trailer region in the 5′-end of the VSV genome. HDV: Hepatitis delta virus ribozyme encoding sequences. T76: Bacteriophage T7 transcriptional terminator sequences. nt: nucleotides. aa: amino acids.

FIG. 2. Construction of recombinant rVSV_{Ind} and rVSV_{NJ} with S1, and RBD+E genes of SARS-CoV-2 with and without honeybee msp and VSV Gtc. Codon-optimized S1 subunit gene and the receptor-binding domain (RBD) of S_{F} plus envelope protein genes of SARS-CoV-2 with and without 21 amino acids honeybee melittin signal peptide [(msp) (SEQ ID NO: 11) NH₂-MKFLVNVALVFMVVYISYIYA-COOH] gene in the diagonal line box, and 49 amino acids VSV G protein transmembrane domain and cytoplasmic tail [(Gtc) (SEQ ID NO: 12) NH₂-SSIASFFFIIGLIIGLFLVLRVGIYLCIKLKHTKKRQIYTDIEMNRLGK-COOH] gene in the horizontal line box were inserted into the G and L gene junction of rVSV_{Ind} and rVSV_{NJ}. In addition, 25-nucleotides-long VSV intergenic junctions [IG, (SEQ ID NO: 13) 5′-CATATGAAAAAAACTAACAGATATC-3′] were inserted between genes to provide transcription termination, polyadenylation and the transcription reinitiation sequences. Recombinant viruses were rescued by VSV reverse genetics.

pT7: Bacteriophage T7 promoter for DNA-dependent RNA polymerase. N: VSV Nucleocapsid Protein gene. P: VSV Phosphoprotein gene. M: VSV Matrix protein gene. G: VSV Glycoprotein gene. L: VSV Large protein, RNA-dependent RNA polymerase gene. l: Leader region in the 3′-end of the VSV genome. t: Trailer region in the 5′-end of the VSV genome. HDV: Hepatitis delta virus ribozyme encoding sequences. T76: Bacteriophage T7 transcriptional terminator sequences. nt: nucleotides. aa: amino acids.

FIG. 3. Expression of SARS-CoV-2 proteins from rVSV_{Ind}-SARS-CoV-2. To check the expression of SARS-CoV-2 RBD, S1, S_{F} (S1+S2), and E proteins from the rVSV_{Ind}-SARS-CoV-2 infected cells, BHK-21 cells were infected with the virus at an MOI of 6. After six hours incubation at 37° C., cell lysates were prepared and protein expression was determined by Western blot analysis. Cell lysates were loaded in 5 μg quantity for SDS-PAGE. RBD, S1, and S_{F} proteins were detected by rabbit antibody against SARS-CoV-2 RBD. S2 protein was detected by rabbit antibody against SARS-CoV-2 S2. E protein was detected by rabbit antibody against SARS-CoV-2 E peptides. (A) Expression of RBD, S1, and S_{F} with and without msp and Gtc. (B) Expression of S2 with and without Gtc. (C) Expression of E protein. (D) Expression of VSV_{Ind} N, P, M, and G proteins. Diagonal line box: honeybee msp, horizontal lines box: VSV Gtc.

FIG. 4. Expression of SARS-CoV-2 proteins from rVSV_{NJ}-SARS-CoV-2. To check the expression of SARS-CoV-2 RBD, S1, and S_{F} (S1+S2) from rVSV_{NJ}-SARS-CoV-2 infected cells, BHK-21 cells were infected with the virus at an MOI of 6. After six hours incubation at 37° C., cell lysates were prepared and protein expression was determined by Western blot. Cell lysates were loaded in 5 μg quantity for SDS-PAGE. RBD, S1, and S_{F} proteins were detected by rabbit antibody against SARS-CoV-2 RBD. S2 protein was detected by rabbit antibody against SARS-CoV-2 S2. E protein was detected by rabbit antibody against SARS-CoV-2 E peptides. (A) Expression of RBD, S1, and S_{F} with and without msp and Gtc. (B) Expression of S2 with and without Gtc. (C) Expression of E protein. (D) Expression of VSV_{Ind} N, P, M, and G proteins. Diagonal line box: honeybee msp, horizontal line box: VSV Gtc.

FIG. 5. SARS-CoV-2 RBD, S1, and S_{F} from recombinant VSV are highly glycosylated. To assess protein glycosylation, 20 μg of infected cell lysates from rVSV_{Ind} (GML) infection (FIG. 3) were treated with 10 units of Peptide N-Glycosidase F (PNGase F, Sigma-Aldrich, G5166) and incubated at 37° C. for 3 hrs. according to the manufacture's protocol. The migratory pattern of the proteins was examined by Western blot analysis. Five μg of the PNGase F treated and untreated cell lysates were loaded on the SDS-PAGE. RBD, S1, and S_{F} were detected by an antibody against SARS-CoV-2 RBD and S2 was detected by an antibody against SARS-CoV-2 S2. (A) Detection of RBD, S1, and S_{F} proteins with and without Gtc in the PNGase F untreated (−) and treated (+) cell lysates. (B) Detection of S2 and S_{F} proteins with and without Gtc in the PNGase F untreated (−) and treated (+) cell lysates. Diagonal line box: honeybee msp, horizontal line box: VSV Gtc.

FIG. 6. Incorporation of RBD with VSV Gtc into rVSV viral particles. Incorporation of SARS-CoV-2 RBD with or without VSV Gtc into rVSV_{Ind} particles and rVSV_{NJ} particles were examined by infecting BHK-21 cells with rVSV-msp-RBD-Gtc+E-Gtc or rVSV-msp-RBD+E at an MOI of 3. The rVSV_{Ind}-SARS-CoV-2 infected cells were incubated at 31° C. for 6 hrs. The rVSV_{NJ}-SARS-CoV-2 infected cells were incubated at 37° C. for 6 hrs. Infected cell lysates were prepared in lysis buffer (lanes 1, 2, and 5). Culture media from the infected cells was centrifuged at 500×g for 10 minutes and supernatant was filtered through a 0.45 μm filter to remove cell debris. The filtered culture media was loaded onto 1 ml of 25% sucrose cushion and ultra-centrifuged at 150,900×g for 3 hrs. Supernatant on top of the 25% sucrose cushion was collected to check the soluble proteins in the media (lanes 3 and 6). Pelleted samples were checked for proteins incorporated into VSV particles (lanes 4 and 7). We detected RBD proteins by Western blot using an antibody against the SARS-CoV-2 RBD protein. (A) Detection of RBD proteins (lanes 1-7) and VSV proteins (lanes 8-14) in cell lysate, concentrated culture media, and virus pellet from cells infected with rVSV_{Ind}-msp-RBD-Gtc+E-Gtc or rVSV_{Ind}-msp-RBD+E. (B) Detection of RBD proteins (lanes 1-7) and VSV proteins (lanes 8-14) in cell lysate, concentrated culture media, and virus pellet from cells infected with rVSV_{NJ}-msp-RBD-Gtc+E-Gtc or rVSV_{NJ}-msp-RBD+E. Diagonal line box: honeybee msp, horizontal line box: VSV Gtc.

FIG. 7. Incorporation of S1 proteins with VSV Gtc into rVSV viral particles. Incorporation of SARS-CoV-2 S1 with or without VSV Gtc into rVSV_{Ind} particles and rVSV_{NJ} particles were examined by infecting BHK-21 cells with rVSV-msp-S1-Gtc or rVSV-S1 at an MOI of 3. The rVSV_{Ind}-SARS-CoV-2 infected cells were incubated at 31° C. for 6 hrs. The rVSV_{NJ}-SARS-CoV-2 infected cells were incubated at 37° C. for 6 hrs. Infected cell lysates were prepared in lysis buffer (lanes 1, 2, and 5). Culture media from the infected cells was centrifuged at 500×g for 10 minutes and supernatant was filtered through a 0.45 μm filter to remove cell debris. The filtered culture media was loaded onto 1 ml of 25% sucrose cushion and ultra-centrifuged at 150,900×g for 3 hrs. Supernatant on top of the 25% sucrose cushion was collected to check the soluble proteins in the media (lanes 3 and 6). Pelleted samples were checked for proteins incorporated into VSV particles (lanes 4 and 7). We detected S1 Western blot using an antibody against the SARS-CoV-2 RBD protein. (A) Detection of S1 proteins (lanes 1-7) and VSV proteins (lanes 8-14) in cell lysate, concentrated culture media, and virus pellet from cells infected with rVSV_{Ind}-msp-S1-Gtc or rVSV_{Ind}-S1. (B) Detection of S1 proteins (lanes 1-7) and VSV proteins (lanes 8-14) in cell lysate, concentrated culture media, and virus pellet from cells infected with rVSV_{NJ}-msp-S1-Gtc or rVSV_{NJ}-S1. Diagonal line box: honeybee msp, horizontal line box: VSV Gtc.

FIG. 8. Incorporation of S_{F} and S2 with VSV Gtc into rVSV_{Ind} viral particles. Incorporation of SARS-CoV-2 S_{F} and S2 with or without VSV Gtc into rVSV_{Ind} particles was examined by infecting BHK-21 cells with rVSV_{Ind}-msp-S_{F}-Gtc or rVSV_{Ind}-S_{F} at an MOI of 3. The rVSV_{Ind}-SARS-CoV-2 infected cells were incubated at 31° C. for 6 hrs. Infected cell lysates were prepared in lysis buffer (lanes 1, 2, and 5). Culture media from the infected cells was centrifuged at 500×g for 10 minutes and supernatant was filtered through a 0.45 μm filter to remove cell debris. The filtered culture media was loaded onto 1 ml of 25% sucrose cushion and ultra-centrifuged at 150,900×g for 3 hrs. Supernatant on top of the 25% sucrose cushion was collected to check the soluble proteins in the media (lanes 3 and 6). Pelleted samples were checked for proteins incorporated into VSV particles (lanes 4 and 7). We detected S1 and S_{F} proteins by Western blot using an antibody against the SARS-CoV-2 RBD protein. S2 and S_{F} proteins were detected by rabbit antibody against SARS-CoV-2 S2. (A) Detection of S_{F} and S1 proteins in cell lysate, concentrated culture media, and virus pellet from cells infected with rVSV_{Ind}-msp-S_{F}-Gtc or rVSV_{Ind}-S_{F}. (B) Detection of S_{F} and S2 proteins in cell lysate, concentrated culture media, and virus pellet from cells infected with rVSV_{Ind}-msp-S_{F}-Gtc or rVSV_{Ind}-S_{F}. (C) Detection of VSV_{Ind} proteins in cell lysate, concentrated culture media, and virus pellet from cells infected with rVSV_{Ind}-msp-S_{F}-Gtc or rVSV_{Ind}-S_{F}. Diagonal line box: honeybee msp, horizontal line box: VSV Gtc.

FIG. 9. Incorporation of S_{F} and S2 with VSV Gtc into rVSV_{NJ} viral particles. Incorporation of SARS-CoV-2 S_{F} and S2 with or without VSV Gtc into rVSV_{NJ} particles was examined by infecting BHK-21 cells with rVSV_{NJ}-msp-S_{F}-Gtc or rVSV_{NJ}-S_{F} at an MOI of 3. The rVSV_{NJ}-SARS-CoV-2 infected cells were incubated at 37° C. for 6 hrs. Infected cell lysates were prepared in lysis buffer (lanes 1, 2, and 5). Culture media from the infected cells was centrifuged at 500×g for 10 minutes and supernatant was filtered through a 0.45 μm filter to remove cell debris. The filtered culture media was loaded onto 1 ml of 25% sucrose cushion and ultra-centrifuged at 150,900×g for 3 hrs. Supernatant on top of the 25% sucrose cushion was collected to check the soluble proteins in the media (lanes 3 and 6). Pelleted samples were checked for proteins incorporated into VSV particles (lanes 4 and 7). We detected S1 and S_{F} proteins by Western blot using an antibody against the SARS-CoV-2 RBD protein. S2 and S_{F} proteins were detected by rabbit antibody against SARS-CoV-2 S2. (A) Detection of S_{F} and S1 proteins in cell lysate, concentrated culture media, and virus pellet from cells infected with rVSV_{NJ}-msp-S_{F}-Gtc or rVSV_{NJ}-S_{F}. (B) Detection of S_{F} and S2 proteins in cell lysate, concentrated culture media, and virus pellet from cells infected with rVSV_{NJ}-msp-S_{F}-Gtc or rVSV_{NJ}-S_{F}. (C) Detection of VSV_{NJ} proteins in cell lysate, concentrated culture media, and virus pellet from cells infected with rVSV_{NJ}-msp-S_{F}-Gtc or rVSV_{NJ}-S_{F}. Diagonal line box: honeybee msp, horizontal line box: VSV Gtc.

FIG. 10. SARS-CoV-2 RBD, S1, S2, and S_{F} with VSV G transmembrane domain and cytoplasmic tail (Gtc) are incorporated efficiently into highly purified rVSV virions. To examine immune responses in mice, it was first necessary to purify rVSV-SARS-CoV-2 viral particles by anion-exchange chromatography. The rVSV-SARS-CoV-2 viral particles were purified as described below. BHK-21 cells grown in T75 flasks were infected with an MOI of 0.1 rVSV-SARS-CoV-2 for 18 hrs in the 31° C. incubator for rVSV_{Ind}-SARS-CoV-2 and in the 37° C. incubator for rVSV_{NJ}-SARS-CoV-2. To remove cellular debris, the culture medium was centrifuged at 4,500 rpm for 5 minutes, and the supernatant was filtered through a 0.45 μm pore size bottle-top filter. The viruses were mixed with 10×SPG buffer to have 2% sucrose, 10 mM potassium phosphate, 0.00376 M KH₂PO₄, 0.0071 M K₂HPO₄, 5 mM glutamate L-glutamic potassium salt monohydrate. The same volume of HNS buffer (10 mM HEPES, 0.465 M NaCl, 2% sucrose) was mixed to prepare the final volume of conditioned virus samples. The conditioned virus samples were purified by anion exchange chromatography using NGC Chromatography System (Bio-Rad) and Mustang Q XT5 membrane column (Pall Canada). The viruses were eluted using a salt gradient formed by mixing equilibration buffer (low salt buffer, pH 7.5: 10 mM HEPES, 0.29 M NaCl, 2% sucrose) and 0% to 70% of elution buffer (high salt buffer, pH 7.0: 10 mM HEPES, 1.5 M NaCl, 2% sucrose). The eluted virus samples were buffer-exchanged with PBS. They were concentrated to about 1 ml volume using a Centricon Plus-70 centrifugal filter device (10K MW cut-off, Millipore). One μg of the purified rVSV-SARS-CoV-2 was analyzed by SDS-PAGE and the presence of RBD, S1, S2, and S_{F} was determined by Western blot analysis. (A) Detection of RBD, S1, and S_{F} on VSV particles. (B) Detection of S2 and S_{F} on VSV particles. (C) Detection of VSV_{Ind} and VSV_{NJ} proteins. (D) Depicted model of pseudotype recombinant VSV virions showing VSV Glycoprotein and SARS-CoV-2 S Glycoprotein (S_{F}, S1, S2 or RBD). rVSV pseudotypes are formed when rVSV-SARS-CoV-2 Spike proteins are expressed with the msp at the NH₂-terminus and VSV Gtc at the COOH-terminus. Diagonal line box: honeybee msp, horizontal line box: VSV Gtc.

FIG. 11. Full-length S_{F} protein with the melittin signal peptide (msp) at the amino terminus and VSV G protein transmembrane domain and cytoplasmic tail (Gtc) at the carboxyl terminus induce the highest IgG titers against the SARS-CoV-2 Spike(ΔTM) protein. For immune response studies, we prime-immunized five C57BL/6 mice (six weeks old) per group with rVSV_{Ind} constructs and boost-immunized with rVSV_{NJ} constructs two weeks after prime immunization. Immunizations were performed intramuscularly on the hind leg. Mice were vaccinated with two different doses, either 5×10⁷ PFU or 5×10⁸ PFU. Two weeks after each immunization, we collected blood from the retro-orbital plexus, and isolated serum from the clotted blood after centrifugation. We incubated serum at 56° C. for 30 minutes and then stored it at −80° C. until further analysis. Serum was collected to determine SARS-CoV-2 S1 protein-specific antibody levels by ELISA on day 13, one day before boost-immunization, and on day 27, two weeks after boost-immunization. We determined the SARS-CoV-2 spike protein-specific IgG levels in mouse sera by indirect ELISA. For ELISA, we coated a 96-well ELISA plate (Thermo Fisher Scientific, Waltham, MA, USA) with 200 ng of S-ATM (LakePharma, CA, USA). We washed the plates with PBST (PBS with 0.05% tween-20) three times and then incubated with blocking buffer (PBS with 2% BSA and 0.05% tween-20) for 1 h at 37° C. We serially diluted serum samples five-fold, starting with a 1:30 dilution in the blocking buffer. We added the diluted serum to the plate and incubated it for two hrs at 37° C. and then washed it three times with PBST. We incubated the plates with 1:3000 diluted HRP-conjugated goat anti-mouse IgG (Southern Biotech, Birmingham, AL, USA) for 1 h at 37° C. and then washed with PBST. We added peroxidase substrate (TMB) solution (Millipore, Billerica, MA, USA) and incubated it for 3 to 5 minutes at room temperature. We stopped the reaction by adding 0.5 N HCl (Merck, Darmstadt, Germany) and OD values were measured at 450 nm using an ELISA plate reader (Molecular Devices, San Jose, CA, USA). We expressed antibody titer as a reciprocal log₁₀ titer of serum dilution showing an OD value of 0.2. (A) Spike(ΔTM)-specific IgG titer after the prime-boost vaccination with doses of 5×10⁷ PFU/mouse (B) Spike(ΔTM)-specific IgG titer after the prime-boost vaccination with doses of 5×10⁸ PFU/mouse. Statistical significance was determined by two-way ANOVA with Tukey's correction (*, p<0.05; **, p<0.005; ***, p<0.001; ns, not significant). The data were presented as means with error bars of standard deviation (n=5 mice per group). Diagonal line box: honeybee msp, horizontal line box: VSV Gtc. VSV-Mock denotes VSV vector alone without any gene insert.

FIG. 12. Full-length S_{F} protein with modifications (rVSV-msp-S_{F}-Gtc) induces the strongest neutralizing antibody titer against SARS-CoV-2. Mice were immunized and sera were collected as described in FIG. 11. SARS-CoV-2 neutralization was determined by FRNT₅₀ assay as described below. For focus reduction assays, immune sera were heat-inactivated at 56° C. for 30 minutes and then serially diluted 2-fold in 25 μl of DMEM with 2% FBS. We mixed the diluted sera with 500 PFU of wild-type SARS-CoV-2 (S Glade, NCCP. 43326, Korea Disease Control and Prevention Agency), in 25 μl per well. We incubated the serum-virus mixture at 37° C. for 30 minutes, after which we added 50 μl of the mixture to Vero cells in a 96-well microplate. We incubated the infected cells at 37° C. with 5% CO₂ for 4 hours and then fixed them with 4% formaldehyde. We treated cells one day after fixation with cold 100% methanol for 10 minutes to increase permeability. We added 100 μl of blocking buffer (1% BSA, 0.5% goat serum, 0.1% tween-20 in PBS) to the wells and mixed the cells with a 1:3000 dilution of primary anti-SARS-CoV-2 NP rabbit mAb (43143-R001, Sino Biological, PA, USA). We incubated the cells at 37° C. for 1 hour. We washed the cells three times with 200 μl of washing buffer (0.1% tween-20 in PBS). We then added goat anti-rabbit IgG-HRP secondary antibody (1721019, Bio-Rad, CA, USA) diluted 1:2000 in blocking buffer to the cells and incubated them for 1-hour at 37° C. We washed the cells three times with wash buffer and added 30 μl of KPL TrueBlue Peroxidase Substrate (Seracare, MA, USA) to the cells for 30 minutes at room temperature. We then counted the developed foci with a spot reader (ImmunoSpot S5, CTL). We counted the number of foci developed in the presence or absence of the diluted serum and then we determined the highest serum dilutions resulting in a 50% reduction in the number of foci. Statistical significance was determined by two-way ANOVA with Tukey's correction (*, p<0.05; ns, not significant). The data were presented as means with error bars of standard deviation. Diagonal line box: honeybee msp, horizontal line box: VSV Gtc.

FIG. 13. The vaccination of hACE2 transgenic mice with rVSV-SARS-CoV-2-msp-S_{F}-Gtc and challenge with SARS-CoV-2. Six-week-old female hACE2 transgenic mice (n=5 per group) were prime-immunized with rVSV_{Ind}-msp-S_{F}-Gtc and boost-immunized with rVSV_{Ind}-msp-S_{F}-Gtc or rVSV_{NJ}-msp-S_{F}-Gtc two weeks after prime-immunization. Four weeks after boost-immunization, mice were challenged intranasally with 1×10⁵ PFU of SARS-CoV-2 (S Glade, National Culture Collection for Pathogens (NCCP) #43326 Korea Disease Control and Prevention Agency) in a 50 μl volume intranasally under anesthesia. The survival and body weight of each mouse was monitored daily.

FIG. 14. Prime-boost vaccination of hACE2 transgenic mice with rVSV-msp-S_{F}-Gtc induces a high level of Spike(ΔTM) specific IgG. Six-week-old female hACE2 transgenic mice were prime vaccinated with rVSV_{Ind}-msp-S_{F}-Gtc and boost vaccinated with rVSV_{Ind}-msp-S_{F}-Gtc or rVSV_{NJ}-msp-S_{F}-Gtc two weeks after the prime-vaccination. Serum was collected to determine the SARS-CoV-2 Spike(ΔTM) protein-specific antibody level by ELISA on day 13, one day before the boost-vaccination and on day 27, two weeks after the boost-vaccination. Statistical significance was determined by two-way ANOVA with Tukey's correction (*, p<0.05; **, p<0.005, ***, p<0.001, ****, p<0.0001; ns, not significant). The data were presented as means with error bars of standard deviation (n=5 mice per group). Diagonal line box: honeybee msp, horizontal line box: VSV Gtc. VSV-Mock denotes VSV vector alone without any gene insert.

FIG. 15. Prime-boost vaccination of hACE2 transgenic mice induces high levels of neutralizing antibodies against SARS-CoV-2. Six-week-old female hACE2 transgenic mice were prime vaccinated with rVSV_{Ind}-msp-S_{F}-Gtc and boost immunized with rVSV_{Ind}-msp-S_{F}-Gtc or rVSV_{NJ}-msp-S_{F}-Gtc two weeks after the prime-vaccination. Serum was collected on day 13, one day before the boost-vaccination and on day 27, two weeks after the boost-vaccination. SARS-CoV-2 neutralization was determined by FRNT₅₀ assay. Statistical significance was determined by two-way ANOVA with Tukey's correction (*, p<0.05; **, p<0.005; ***, p<0.001; ns, not significant). The data were presented as means with error bars of standard deviation (n=5 mice per group). Diagonal line box: honeybee msp, horizontal line box: VSV Gtc. VSV-Mock denotes VSV vector alone without any gene insert.

FIG. 16. Bodyweight and survival of the vaccinated and SARS-CoV-2 challenged hACE2 transgenic mice. Six-week-old female hACE2 transgenic mice (n=5 per group) were prime-vaccinated with rVSV_{Ind}-msp-S_{F}-Gtc and boost vaccinated with rVSV_{Ind}-msp-S_{F}-Gtc or rVSV_{NJ}-msp-S_{F}-Gtc two weeks after prime-vaccination. Four weeks after boost-vaccination (FIG. 13), mice were challenged intranasally with 1×10⁵ PFU of SARS-CoV-2. The survival and body weight of each mouse was monitored daily. (A) Average bodyweights of mice in each vaccinated group. (B) Individual body weights for mice vaccinated with rVSV-Mock and challenged with SARS-CoV-2. (C) Mouse survival after SARS-CoV-2 challenge. VSV-Mock denotes VSV vector alone without any gene insert.

FIG. 17. SARS-CoV-2 viral loads in the lungs of vaccinated and challenged hACE2 transgenic mice. Human ACE2 transgenic mice were vaccinated and challenged with SARS-CoV-2 as described in FIG. 13. Right lobes of mice lungs were aseptically removed from the mice on day 3, day 7, and day 15 after SARS-CoV-2 challenge. Infectious SARS-CoV-2 was quantified by plaque assay on Vero E6 cells. Statistical significance was determined by two-way ANOVA with Tukey's correction (****, p<0.0001). VSV-Mock denotes VSV vector alone without any gene insert.

FIG. 18. Histopathological findings in the lungs of hACE2 mice 3 days after the SARS-CoV-2 challenge. Human ACE2 transgenic mice were vaccinated and challenged with SARS-CoV-2 as described in FIG. 13. Left lobes of mice lungs were fixed in 10% buffered formalin on day 3, day 7, and day 16 after the SARS-CoV-2 challenge. Lung tissues were processed and embedded in low-melting paraffin, sectioned to a thickness of 3 μm, and stained with hematoxylin and eosin. Stained tissues were examined under a light microscope (Olympus CS41, Japan) with 100× magnification. Note: a, alveolus; b, bronchiole; v, blood vessels. Arrows show infiltration of inflammatory cells (lymphocytes and macrophages). G1: empty vector infected mice, G2: 5×10⁸ of rVSV_{Ind}-msp-S_{F}-Gtc/rVSV_{NJ}-msp-S_{F}-Gtc vaccinated mice, G3: 5×10⁸ of rVSV_{Ind}-msp-S_{F}-Gtc/rVSV_{Ind}-msp-S_{F}-Gtc vaccinated mice, G4: 5×10⁷ of rVSV_{Ind}-msp-S_{F}-Gtc/rVSV_{NJ}-msp-S_{F}-Gtc vaccinated mice, G5: uninfected mice.

FIG. 19. Histopathological findings in the lungs of hACE2 mice 7 days after the SARS-CoV-2 challenge. Human ACE2 transgenic mice were vaccinated and challenged with SARS-CoV-2 as described in FIG. 13. Left lobes of mice lungs were fixed in 10% buffered formalin on day 3, day 7, and day 16 after the SARS-CoV-2 challenge. Lung tissues were processed and embedded in low-melting paraffin, sectioned to a thickness of 3 μm, and stained with hematoxylin and eosin. Stained tissues were examined under a light microscope (Olympus CS41, Japan) with 100× magnification. Note: a, alveolus; b, bronchiole; v, blood vessels. Arrows show infiltration of inflammatory cells (lymphocytes and macrophages). G1: empty vector infected mice, G2: 5×10⁸ of rVSV_{Ind}-msp-S_{F}-Gtc/rVSV_{NJ}-msp-S_{F}-Gtc vaccinated mice, G3: 5×10⁸ of rVSV_{Ind}-msp-S_{F}-Gtc/rVSV_{Ind}-msp-S_{F}-Gtc vaccinated mice, G4: 5×10⁷ of rVSV_{Ind}-msp-S_{F}-Gtc/rVSV_{NJ}-msp-S_{F}-Gtc vaccinated mice, G5: uninfected mice.

FIG. 20. Histopathological findings in the lungs of hACE2 mice 16 days after the SARS-CoV-2 challenge. Human ACE2 transgenic mice were vaccinated and challenged with SARS-CoV-2 as described in FIG. 13. Left lobes of mice lungs were fixed in 10% buffered formalin on day 3, day 7, and day 16 after the SARS-CoV-2 challenge. Lung tissues were processed and embedded in low-melting paraffin, sectioned to a thickness of 3 μm, and stained with hematoxylin and eosin. Stained tissues were examined under a light microscope (Olympus CS41, Japan) with 100× magnification. Note: a, alveolus; b, bronchiole; v, blood vessels. Arrows show infiltration of inflammatory cells (lymphocytes and macrophages). G1: empty vector infected mice, G2: 5×10⁸ of rVSV_{Ind}-msp-S_{F}-Gtc/rVSV_{NJ}-msp-S_{F}-Gtc vaccinated mice, G3: 5×10⁸ of rVSV_{Ind}-msp-S_{F}-Gtc/rVSV_{Ind}-msp-S_{F}-Gtc vaccinated mice, G4: 5×10⁷ of rVSV_{Ind}-msp-S_{F}-Gtc/rVSV_{NJ}-msp-S_{F}-Gtc vaccinated mice, G5: uninfected mice.

FIG. 21. Neutralization of wild-type and variants of concern (VOCs) SARS-CoV-2 by a monoclonal anti-S protein antibody and by sera from macaques prime/boost immunized with rVSV-msp-SARS-CoV-2-Gtc. Macaques were prime-immunized intramuscularly with 10 9 PFU rVSV_{Ind}-SARS-CoV-2-msp-S_{F}-Gtc and boost-immunized with 10 9 PFU of rVSV_{Ind}-SARS-CoV-2-msp-S_{F}-Gtc 20 days after the prime-immunization. Sera were collected on day 14 after boost-immunization. For measuring neutralization by the monoclonal Sino-R001 NAb and in sera of macaques immunized with prime-boost with rVSV-msp-S_{F}-Gtc, monoclonal Nab and sera from macaque B was serially diluted two-fold starting at 1:40 and added to Vero E6 cells along with 100 plaque-forming units (PFU) of SARS-CoV-2 wild type (panel A for NAb and panel E for Rhesus macaque B), SARS-CoV-2 Alpha variant (panel B and F), SARS-CoV-2 Beta variant (panel C and G), or SARS-CoV-2 Gamma variant (panel D and H). The monoclonal Sino-R001 NAb was only diluted to 1/640. All assays were performed in quadruplicate. For the neutralizing assays using SARS-CoV-2 wild-type and variants, qRT-PCR was performed on the viral RNA released into the supernatant from the cells exposed to the sera and converted to % neutralization based on maximal replication in absence of sera. All RNA extracts were collected using a QIAamp 96 Viral RNA Kit (Qiagen, MD, US) following the manufacturers protocol. The titer resulting in 50% neutralization was based on virus production measured by qRT-PCR. qRT-PCR reactions were performed on a QuantStudio 5 Real-Time PCR system (Applied Biosystems, Waltham, MA, US) with TaqMan Fast Virus 1-Step reagents (Applied Biosystems, Waltham, MA, US) following the manufacturer's protocol. In panels E through H, the level of neutralization of heat-inactivated preimmunization sera from macaque B was also run and shown on the graph for only the 1/40, 1/80, and 1/160 dilutions. Cross neutralization of alpha, beta, and gamma variants of SARS-CoV-2 by antisera against Wuhan strain showed that macaque's anti-Wuhan antibody neutralizes 100% of the original Wuhan strain and alpha variants, approximately 80% of beta variants, and approximately 55% of gamma variants.

FIG. 22. Construction of rVSV-msp-S_{F}-Gtc with mutations from various SARS-CoV-2 variants, Beta, Delta, and Omicron variants. Mutations of K417N (aag to aat), E484K (gag to aag), and N501Y (aac to tac) were introduced to the S_{F} of msp-S_{F}-Gtc to generated vaccines for SARS-CoV-2 Beta variants. Mutations of T95I (acc to atc), G142D (ggc to gac), E156G (gag to ggc), Del 157/158 (VT, gtgtat), A222V (gcc to gtg), L452R (ctg to cgg), T478K (acc to aag), D614G (gac to ggc), P681R (ccc to cgg), D950N (gat to aac) were introduced to the S_{F} of msp-S_{F}-Gtc to generated vaccines for SARS-CoV-2 Delta variants. Mutations of A67V (gcc to gtg), Del 69/70 (HV, cacgtg), T95I (acc to atc), G142D (ggc to gac), Del 143-145 (VYY, gtgtactat), N211I (aac to atc), Del 212 (L, ctg), Ins 214 EPE (gagcccgag), G339D (ggc to gac), S371L (tcc to ctg), S373P (tct to ccc), S375F (agc to ttc), K417N (aag to aac), N440K (aat to aag), G4465 (ggc to agc), S477N (agc to aac), T478K (acc to aag), E484A (gag to gcc), Q493R (cag to cgg), G4965 (ggc to agc), Q498R (cag to cgg), N501Y (aac to tac), Y505H (tat to cac), T547K (acc to aag), D614G (gac to ggc), H655Y (cac to tac), N679K (aac to aag), P681H (ccc to cac), N764K (aat to aag), D796Y (gac to tac), N856K (aat to aag), Q954H (cag to cac), N969K (aac to aag), and L981F (ctg to ttc) were introduced to the S_{F} of msp-S_{F}-Gtc to generated vaccines for SARS-CoV-2 Omicron variants. The msp-S_{F}-Gtc with the mutations were cloned into prVSV_{Ind} (GML) and prVSV_{NJ} (GMM) vectors, and the viruses were recovered from the DNA by VSV reverse genetics. rVSVs carrying the spike protein genes of beta, delta, and Omicron variants have been recovered and cross neutralization of all variants are being investigated.

### DETAILED DESCRIPTION OF THE INVENTION

### 1. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this disclosure belongs.

The articles “a” and “an” are used herein to refer to one or more than one of the grammatical object of the article.

“And/or” when used between objects, is used to refer to at least one of the objects. For example, “the S (full or partial length forms) and/or the E proteins” is used to refer to only the S protein, to only the E protein, or to both the S protein and the E protein.

The terms “animal” and “subject” as used herein includes all members of the animal kingdom including mammals, preferably humans.

The term “effective amount” as used herein means an amount effective and at dosages and for periods of time necessary to achieve the desired result.

“rVSV” is used to refer to a recombinant vesicular stomatitis virus.

The term “Indiana”, and “Ind” are used to refer to the Indiana (VSV_{Ind}) serotype of VSV. The term “New Jersey”, and “NJ” are used to refer to the New Jersey (VSV_{NJ}) serotype of VSV.

“M_{WT}” “M(WT)” are used to refer to VSV having a wild type M gene (SEQ ID NO: 1 (nucleotide sequence of M gene of VSV_{Ind}) Table 7; SEQ ID NO: 3 (amino acid sequence of wildtype M protein of VSV_{Ind}) Table 8); SEQ ID NO: 5 (nucleotide sequence of wildtype M protein of of VSV_{NJ}) Table 9 and SEQ ID NO: 8 (amino acid sequence of M protein of VSV_{NJ}) Table 10).

“G22E” is used to refer to an M_{WT} gene in VSV_{NJ} having a glycine changed to a glutamic acid at position 22.

“G21E” is used to refer to an M_{WT} gene in VSV_{Ind} having a glycine changed to a glutamic acid at position 21.

“L111A” is used to refer to an M_{WT} gene in VSV_{Ind} having a leucine changed to alanine at position 111.

“M51R” is used to refer to an M_{WT} gene in the VSV_{Ind} having a methionine changed to an arginine at position 51.

“M48R+M51R” or “M48R/M51R” are used to refer to an M_{WT} gene in VSV_{NJ} having a methionine changed to an arginine at positions 48 and 51 respectively.

“rVSV_{Ind} (GML)” is used to refer to an M_{WT} gene in VSV_{Ind} having the combined mutation G21E, M51R and one of L111A or L111F.

“rVSV_{NJ} (GMM)” is used to refer to an M_{WT} gene in VSV_{NJ} having the combined mutation G22E, M48R/M51R.

“mspRBD” is a recombinant receptor binding domain in the S protein of SARS-CoV-2 (RBD) having a honeybee melittin signal peptide (msp) at the NH₂ terminus of the RBD.

“msp-RBD-Gtc+E-Gtc” is a recombinant RBD having the msp at the NH₂-terminus of the RBD, a VSV G protein transmembrane domain and cytoplasmic tail (Gtc) at the C-terminus of the RBD and an E protein of SARS-CoV-2 having Gtc at the C-terminus.

“msp-RBD-Gtc+E” is a recombinant RBD having the msp at the NH₂-terminus of the RBD, a VSV G protein transmembrane domain and cytoplasmic tail (Gtc) at the C-terminus of the RBD and an E protein of SARS-CoV-2.

“msp-S_{F}-Gtc” is a recombinant full length S protein of SARS-CoV-2 having the signal peptide sequence of wild type S protein replaced with a msp at the NH₂-terminus of the S protein and a VSV G protein transmembrane domain and cytoplasmic tail (Gtc) at the C-terminus.

“msp-S1-Gtc” is a recombinant S1 region of S protein of SARS-CoV-2, having the signal peptide sequence of wild type S1 peptide replaced with a honeybee melittin peptide (msp) at the NH₂-terminus of the S1 peptide and a VSV G protein transmembrane domain and cytoplasmic tail (Gtc) at the C-terminus of the S1 peptide.

“RBD” is used to refer to the receptor binding domain of the S_{F}.

“S” is a recombinant S1 region of S_{F} of SARS-CoV-2.

“S2” is a recombinant S2 region of S_{F} of SARS-CoV-2.

“S_{F}” is a recombinant full length S protein of SARS-CoV-2.

“S protein” is used to refer to the S_{F} or partial length forms of the spike protein of SARS-CoV-2.

“Partial length of the S protein” is used to refer to one or more of S1, S2 and RBD.

The term “protein” as used herein is defined as a chain of amino acid residues, usually having a defined sequence. As used herein the term protein is inclusive of the terms “peptides” and “proteins”. The terms also encompass an amino acid polymer that has been modified.

### 2. Overview

The present disclosure features rVSVs, vaccines, prime boost immunization combinations, immunization platforms, immunization regimens and medicaments and kits useful for inducing an immune response in a subject and preventing SARS-CoV-2 (including wild type SARS-CoV-2 and SARS-CoV-2 variants) infection in a subject, wherein said rVSVs, vaccines, prime boost immunization combinations, platforms, regimens and medicaments and useful kits comprise a rVSV that carries one or more genes that encode for a S protein of SARS-CoV-2, or the E protein of SARS-CoV-2, or both the S protein and the E protein, including one or more modifications of the S and E proteins. In embodiments, the one or more genes that encode for the S and/or E proteins are codon-optimized for expression in a human cell. The rVSV may be rVSV_{Ind} or rVSV_{NJ}, including their different strains, such as Hazelhurst strain (VSV_{NJ-H}) and or Ogden strain (VSV_{NJ-O}). “SARS-CoV-2” include wild type SARS-CoV-2 and SARS-CoV-2 variants. Non-exhaustive list of SARS-CoV-2 variants include variants Alpha, Beta, Delta, Gamma, Epsilon, Eta, Iota, Kappa, 1.617.3, Mu, Omicron, Zeta and their respective descendent lineages.

The S protein of SARS-CoV-2 can be a full-length spike (S_{F}) protein or a partial length S protein. The partial length form of the S protein is one or more of a S1 peptide of the S_{F} protein, a S2 peptide of the S_{F} protein, a receptor binding domain of the S_{F} protein (RBD) or any modifications thereof.

In embodiments, at least one of the S protein and the E protein are modified with a honeybee melittin signal peptide (msp) in the NH₂ terminus of the at least one of the S protein and the E protein, and/or a VSV G protein transmembrane domain and cytoplasmic tail (Gtc) at the C-terminus of the at least one of the S protein and the E protein.

### 3. Vaccines or Immunogenic Compositions

The present disclosure further features vaccines or immunogenic compositions.

This disclosure describes SARS-CoV-2 vaccines or immunological compositions including a recombinant vesicular stomatitis virus (rVSV) that carries one or more genes that encode for the S (full or partial length forms) and/or the E proteins of SARS-CoV-2, including modifications of said S and E proteins. The S protein can be provided as a full-length spike (S_{F}) protein, a S1 peptide of the S_{F} protein, a S2 peptide of the S_{F} protein, and/or a receptor binding domain of the S_{F} protein (RBD). In embodiments, the at least one of the S and E proteins are modified with a honeybee melittin signal peptide (msp) in its NH₂ terminus and/or a VSV G protein transmembrane domain and cytoplasmic tail (Gtc) at the C-terminus of the S and/or E protein. In embodiments, the signal peptide of wild type S_{F} is replaced with the msp. In embodiments, one or more genes that encode for the S and E proteins are codon-optimized for expression in a human cell. The rVSV may be of Indiana serotype, New Jersey serotype or any other suitable VSV serotype.

This disclosure also provides for prime-boost immunization regimens. A prime boost immunization combination against SARS-CoV-2 include: (a) a prime vaccine or immunogenic composition comprising a replication competent recombinant vesicular stomatitis virus (rVSV) carrying one or more genes that encode for at least one of the S protein and the E protein of a SARS-CoV-2, and (b) a booster vaccine or immunogenic composition comprising a replication competent rVSV carrying the same one or more genes that encode for the at least one of the S and E proteins of SARS-CoV-2. The S protein of SARS-CoV-2 is one or more of a full-length spike (S_{F}) protein, a S1 peptide of the S_{F} protein, a S2 peptide of the S_{F} protein, and/or a receptor binding domain of the S_{F} protein (RBD). In embodiments, at least one of the S protein and E protein is modified with a honeybee melittin signal peptide (msp) in its NH₂ terminus and/or a VSV G protein transmembrane domain and cytoplasmic tail (Gtc) at the C-terminus of the S protein and/or E protein. In embodiments, the one or more genes that encode for the at least one of the S and E proteins are codon-optimized for expression in a human cell.

The rVSV of the prime and boost vaccines may be of the same or different serotypes. In one example, the prime vaccine or immunological composition carries a rVSV_{Ind} according to the present disclosure and the boost vaccine or immunological composition carries a rVSV_{Ind} or a rVSV_{NJ} according to the present disclosure. In another example, the prime vaccine or immunological composition carries a rVSV_{NJ} according to the present disclosure and the boost vaccine carries a rVSV_{Ind} or a rVSV_{NJ} according to the present disclosure.

The vaccine or immunogenic compositions of the disclosure are suitable for administration to subjects in a biologically compatible form in vivo. The expression “biologically compatible form suitable for administration in vivo” as used herein means a form of the substance to be administered in which any toxic effects are outweighed by the therapeutic effects. The substances maybe administered to any animal or subject, preferably humans. The vaccines of the present disclosure may be provided as a lyophilized preparation. The vaccines of the present disclosure may also be provided as a solution that can be frozen for transportation. Additionally, the vaccines may contain suitable preservatives such as human albumin, bovine albumin, sucrose, glycerol or may be formulated without preservatives. If appropriate (i.e. no damage to the VSV in the vaccine), the vaccines may also contain suitable diluents, adjuvants and/or carriers.

The dose of the vaccine may vary according to factors such as the disease state, age, sex, and weight of the individual, and the ability of antibody to elicit a desired response in the individual. Dosage regime may be adjusted to provide the optimum therapeutic response. The dose of the vaccine may also be varied to provide optimum preventative dose response depending upon the circumstances.

### 4. Methods of Use

The present disclosure also features methods of inducing an immune response in a subject against SARS-CoV-2 and/or preventing or treating a SARS-CoV-2 infection in a subject comprising administering to the subject an effective amount of a combination of vaccines or immunogenic compositions of the present disclosure.

As such, in one aspect, the present disclosure provides for a method for inducing an immune response in a subject to a SARS-CoV-2 characterized in that said method comprises the step (a) of administering to the subject an effective amount of a vaccine or immunogenic composition including a rVSV of a serotype carrying one or more genes that encode for at least one of the S protein and the E protein of SARS-CoV-2. In one embodiment, the method further comprises the step (b) of administering to the subject a boost vaccine or immunogenic composition comprising a rVSV of the same serotype as in step (a) or of another serotype (i.e., a serotype different from the one used in the vaccine or immunogenic composition used in step (a)) carrying the same one or more genes that encode for the at least one of the S and E proteins of SARS-CoV-2. In embodiments, the one or more genes that encode for the at least one of S and E proteins are codon-optimized for expression in a human cell. In embodiments, the boost vaccine of step (b) is followed by another booster vaccine(s) or immunogenic composition(s) (second, third, fourth and so forth boosters). The booster vaccine(s) or immunogenic composition(s) comprising a rVSV of the same serotype as in step (a) or of another serotype (i.e., a serotype different from the one used in the vaccine or immunogenic composition used in step (a)) carrying the same one or more genes that encode for the at least one of the S and E proteins of SARS-CoV-2.

The S protein of SARS-CoV-2 is one or more of a full-length spike (S_{F}) protein, a S1 peptide of the S_{F} protein, a S2 peptide of the S_{F} protein, and/or a receptor binding domain of the S_{F} protein (RBD). In embodiments, at least one of the S protein and E protein is modified with a honeybee melittin signal peptide (msp) in its NH₂ terminus and/or a VSV G protein transmembrane domain and cytoplasmic tail (Gtc) at its C-terminus.

In aspects of the disclosure the methods for inducing an immune response in a mammal to a SARS-CoV-2 and the methods for preventing or treating an infection caused by SARS-CoV-2 may further comprise the step of (c) administering to the subject an effective amount of the vaccine or immunogenic composition of either step (a) or step (b). Step (c) may be administered to the subject more than one time over the course of inducing an immune response, preventing or treating.

The above disclosure generally describes the present disclosure. A more complete understanding can be obtained by reference to the following specific Examples. These Examples are described solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitation.

### Examples

The examples are described for the purposes of illustration and are not intended to limit the scope of the invention.

### 1. rVSV-SARS-CoV-2 Vaccine Constructs

Previously, we have developed attenuated recombinant vesicular stomatitis viruses (rVSV) as vaccine vectors using two antigenically distinct serotypes (4), the Indiana serotype (rVSV_{Ind}) and the New Jersey serotype (rVSV_{NJ}). There are two different strains of New Jersey serotype, Hazelhurst strain (VSV_{NJ-H}) and Ogden strain (VSV_{NJ}-0). We have used both strains of New Jersey serotype depending on which strain produces higher recombinant virus titer.

To generate safe vaccine vectors, we combined the temperature-sensitive mutation (L111A) with the M51R mutation in the M gene to attenuate the pathogenicity of rVSV_{Ind} (4). The G21E mutation in the M gene of rVSV_{Ind} did not have any effect on the temperature sensitivity of the rVSV_{Ind}, but the combined mutations, G21E, M51R, and L111A (GML) attenuated further from the double mutations of M51R+L111A in the M gene of rVSV_{Ind}. The resulting attenuated temperature-sensitive rVSV_{Ind} is rVSV_{Ind} (GML). The M gene mutant, rVSV_{NJ} (GMM) contains G22E, M48R, and M51R mutations. We found that the combined mutations of G22E, M48R, and M51R in the New Jersey serotype M gene significantly reduced cytopathogenicity of VSV_{NJ} (4, 5).

Structurally, SARS-CoV-2 has four main structural proteins including spike (S) glycoprotein, small envelope (E) glycoprotein, membrane (M) glycoprotein, and nucleocapsid (N) protein, and also several accessory proteins (6).

The SARS-CoV-2 Spike (S) protein is cleaved into S1 and S2. The S proteins on the surface of the SARS-CoV-2 particle binds to human angiotensin-converting enzyme 2 (hACE2) on the cell surface through the receptor-binding domain (RBD) in the S1 region (7, 8). The S2 with the transmembrane and cytoplasmic tail at the carboxyl terminus anchors the S to the SARS-CoV-2 envelope. Therefore, these parts of full-length S have important roles in the entry of the SARS-CoV-2 to the host cell to replicate and they are favorable targets for vaccine development. The antibody raised against RBD, S1, or full-length S will bind to the proteins, neutralize the viruses, and will block the entry of the viruses to the host cells.

There are 61 codons in nature to code for 20 amino acids. Certain codons are utilized preferentially in different organisms, which means certain organisms are biased for certain codons. Therefore, if the viral genes, which are not expressed normally in humans are to be expressed in humans, it would be better to optimize the codon usage in humans to express the protein more efficiently. Therefore, we codon-optimized RBD, E1, S1, and full-length S of SARS-CoV-2 (GenBank: JX869059.2) for human usage through Genscript USA Inc (Piscataway, NJ, USA). The codon-optimized genes were cloned into avirulent rVSV full-length clones (prVSV_{Ind} (GML) and prVSV_{NJ} (GMM)) with and without the modification of glycoprotein signal peptides at NH₂-terminus and the replaced the transmembrane and cytoplasmic tail (Gtc) of VSV G protein at COOH terminus.

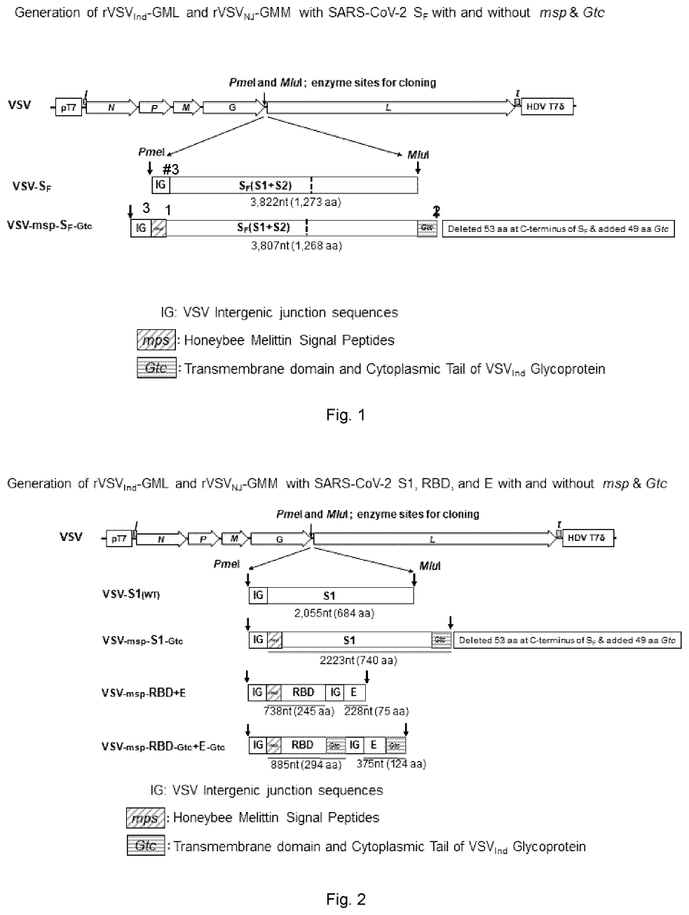

Signal peptides at the amino-terminal region of the secretory proteins target the protein to the ER and Golgi network for the modification of the protein and to the cytoplasmic membrane for the secretion. Honeybee melittin signal peptide (msp) increases the overall expression level, glycosylation, and secretion of the protein through the cytoplasmic membrane (9). Therefore, we replaced the signal peptide sequences of SARS-CoV-2 full-length S and S1 with msp and added the msp to the NH₂ terminus of the RBD to increase the expression, glycosylation, and secretion of the proteins. (FIGS. 1 and 2). The carboxyl-terminal region of the transmembrane and cytoplasmic tail (tc) of the full-length S helps the protein to localize on the envelope to produce infectious SARS-CoV-2 particles. Addition or replacing the tc of SARS-CoV-2 S with that of VSV G protein Gtc (FIGS. 1 and 2) will help the SARS-CoV-2 S localize on the VSV particles and will result in the pseudotyped VSV. The combination of changes made to the RBD, S1, and full-length S are made to enhance the CD8⁺ and CD4⁺ T cell responses as well as humoral immune responses. We inserted the genes of both modified and unmodified RBD+E, S1, and full-length S into the G gene and L gene of prVSV_{Ind} (GML) and prVSV_{NJ} (GMM) at Pme I and Mitt I sites (FIGS. 1 and 2). The recombinant viruses were recovered by VSV reverse genetics. The viruses were purified by 3 consecutive plaque picking and stock viruses were prepared by infecting BHK21 cells.

### 2. Expression of SARS-CoV-2 Proteins from rVSV_{Ind} (GML) and rVSV_{NJ} (GMM)

To check the expression of SARS-CoV-2 RBD plus E, S1, and full-length S from the rVSV_{Ind} (GML), we infected BHK21 cells with an MOI of 6, incubated the cells in the 37° C. CO₂ incubator for 6 hours, cell lysates were prepared in the lysis buffer. The expression level of the proteins was determined by Western blot analysis. The cell lysates were loaded in 5 μg—for the SDS-PAGE. RBD and S1 were detected by the rabbit antibody against SARS-CoV-2 RBD (Sino Biological, 40592-T62). S2 protein was detected by the rabbit antibody against SARS-CoV-2 S2 (Sino Biological, 40590-T62).

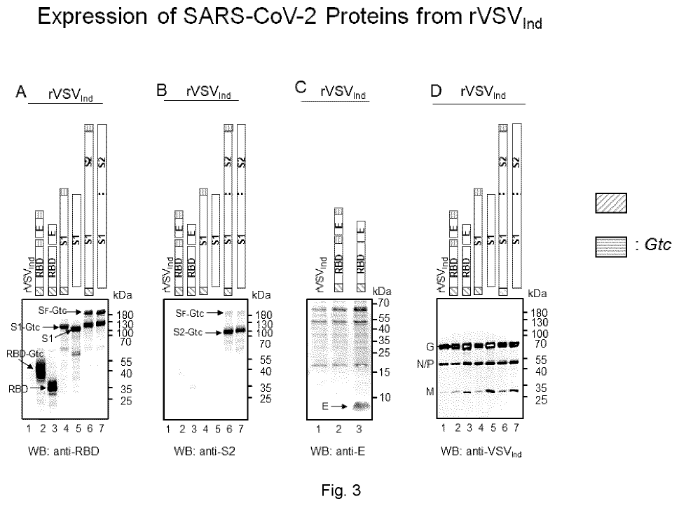

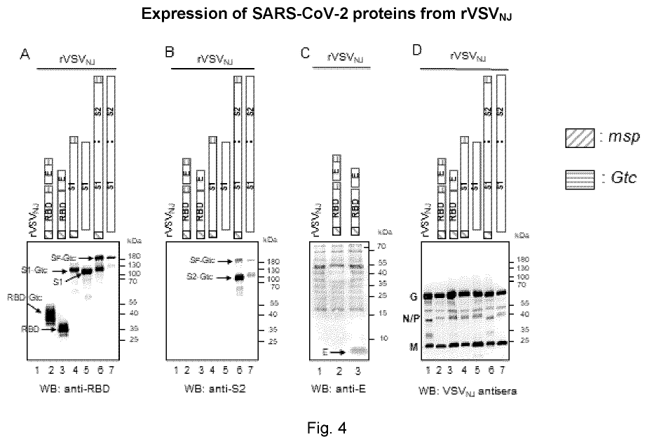

RBD, S1, and S2 proteins were expressed in good quantities from rVSV_{Ind} (GML) (FIGS. 3A and 3B). The addition of VSV Gtc to RBD (FIG. 3A lane 2) caused the protein to migrate slower than the RBD without the Gtc (FIG. 3A lane 3). RBD without Gtc is supposed to migrate as 26.9 kDa protein and RBD-Gtc supposed to migrate as a 32.4 kDa protein. Both RBD with and without the Gtc migrated as larger proteins than the predicted molecular masses of the proteins, which indicated the heavy glycosylation of the proteins (FIG. 3A lane 2 and 3). The predicted molecular masses of msp-S1-Gtc and S1 are 81 kDa and 75.35 kDa, respectively. These proteins also migrated slower than the predicted sizes of the proteins (FIG. 3A lanes 4, 5). Expression of full-length S produced full-length S and S1 proteins (FIG. 3A lane 6 and 7) and S2 (FIG. 3B lane 6 and 7), which demonstrated that full-length S is cleaved into S1 and S2. A similar amount of VSV proteins were detected in all cell lysates (FIG. 3D).

RBD, S1, and full-length S with and without msp and Gtc from rVSV_{NJ} (GMM) expressed good quantities except for the full-length S from the rVSV_{NJ} (GMM)-S (FIG. 4A, lane 7 and FIG. 4B, lane 7). The proteins expressed from the rVSV_{NJ} (GMM) also showed the same migration pattern on the SDS-PAGE gel as those from the rVSV_{Ind} (GML). We could detect similar quantities of G and M proteins of rVSV_{NJ} clearly in all cell lysates (FIG. 4D).

### 3. SARS-CoV-2 RBD, S1, and S from rVSV are Highly Glycosylated

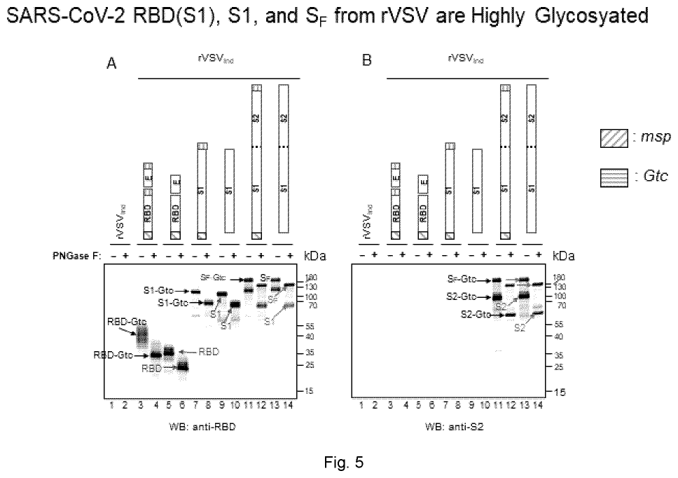

The proteins expressed from rVSV were migrated slower than the predicted molecular masses, which was calculated based on the number of amino acids (FIG. 3 and FIG. 4). To confirm that the slow migration of the proteins was caused by the heavy glycosylation of the proteins we treated lysates of rVSV_{Ind} (GML)-SARS-CoV-2-infected cells. We treated 20 μg of infected cell lysates with 10 units of Peptide N-Glycosidase F (PNGase F, Sigma-Aldrich, G5166) and incubated them at 37° C. for 3 hrs. according to the manufacture's protocol. The migratory pattern of the proteins was examined by Western blot analysis. Five μg of the PNGase F treated and untreated cell lysates were loaded on the SDS-PAGE gel. RBD, S1, and full-length S was detected by an antibody against SARS-CoV-2 RBD and S2 was detected by an antibody against SARS-CoV-2 S2 (FIG. 5).

PNGase F treated RBD (FIG. 5A lane 4 and 6), S1 (FIG. 5A, lane 8 and 10), full-length S (FIG. 5A and B, lane 12 and 14), and S2 (FIG. 5B lane 12 and 14) migrated faster than the proteins, which were not treated with PNGase F. The results confirmed that SARS-CoV-2 S proteins expressed from the rVSVs are highly glycosylated as the S proteins from the SARS-CoV-2.

### 4. Addition of Transmembrane and Cytoplasmic Tail of VSV_{Ind} Glycoprotein (Gtc) to the Carboxyl Terminus of SARS-CoV-2 Proteins Make the Proteins Incorporated Efficiently into the rVSV Particles

Adding honeybee melittin signal peptides (msp) to the secretory proteins increases the overall expression level, glycosylation, and secretion of the protein through the cytoplasmic membrane. Placing the transmembrane domain (TM) and cytoplasmic tail (CT) of VSV glycoprotein (G) at the carboxyl terminus of SARS-CoV-2 RBD, S1, and full-length S protein will make the proteins incorporated in the membrane of VSV particles. Therefore, adding honeybee msp at the NH₂ terminus and TM and CT of VSV G (Gtc) at the COOH terminus in the SARS-CoV-2 S protein will make the protein secreted in better quantities and also localize in the VSV particles. The presence of SARS-CoV-2 S proteins on the surface of VSV will present the proteins to the immune cells as a virus surface antigen as well as a newly produced and secreted form of S proteins.

To check the incorporation of SARS-CoV-2 RBD, S1, and full-length S into rVSV_{Ind} (GML) and rVSV_{NJ} (GMM) particles we infected BHK21 cells with an MOI of 3 of rVSV_{Ind} (GML)-SARS-CoV-2 or rVSV_{NJ} (GMM)-SARS-CoV-2. The cells infected with rVSV_{Ind} (GML)-SARS-CoV-2 were incubated at 31° C. for 6 hrs. The cells infected with rVSV_{NJ} (GMM)-SARS-CoV-2 were incubated at 37° C. for 6 hrs. Culture media from the infected cells were centrifuged for 10 minutes at 4,500 rpm and were filtered through the 0.45 μm filter to remove cell debris. The filtered culture media was loaded onto the 25% sucrose cushion and was ultra-centrifuged for 3 hrs at 35,000 rpm. The supernatant on top of the 25% sucrose was collected to check the proteins in the media, which was not incorporated into the VSV particles. The collected supernatant was concentrated down to 200 μl volume from 10 ml volume using a 10K molecular weight cut-off size centrifugal filter unit called Amicon Ultra-15 (Merck Millipore Ltd, UFC901024). The pellet was resuspended in 200 μl of PBS and was used to check the proteins incorporated into the VSV particles. The SARS-CoV-2 proteins in the cell lysates, in the concentrated supernatant, and the pellets were checked by Western blot analysis.

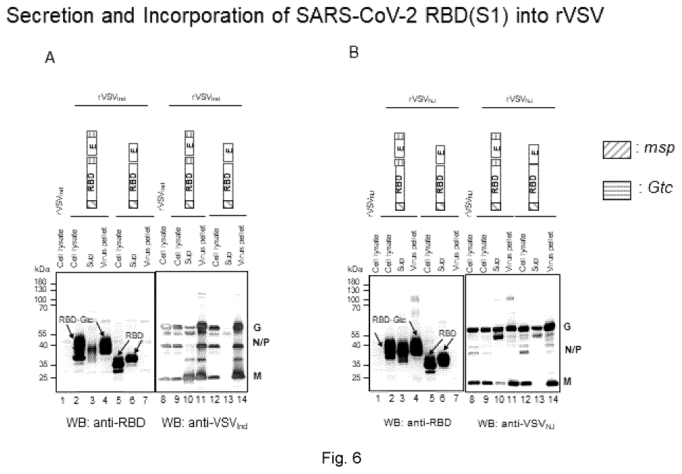

RBD proteins with and without the Gtc were detected in the cell lysate and the concentrated culture media from the cells infected with rVSV_{Ind} (GML)-msp-RBD-Gtc+E-Gtc, rVSV_{Ind} (GML)-msp-RBD+E and rVSV_{NJ} (GMM)-msp-RBD-Gtc+E-Gtc, and rVSV_{NJ} (GMM)-msp-RBD+E (FIGS. 6A and 6B, lanes 2, 3, 5, and 6) indicating that they could secret from the cells, but only RBD with Gtc could incorporate into the VSV particles (FIGS. 6A and 6B, lane 4). The result indicated that the RBD with msp could secret but it required VSV Gtc to be able to incorporate into the VSV particles.

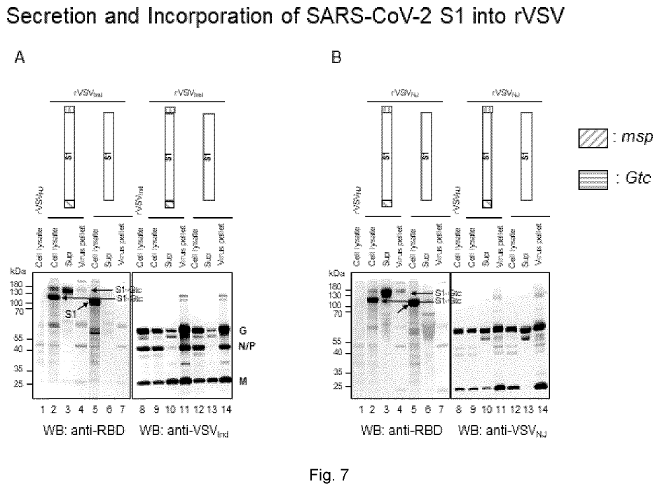

The importance of msp to make the protein secreted from the infected cells was demonstrated by S1 protein. S1 without the modifications at both NH₂ and COOH termini was not detected in both concentrated supernatant and in the pellet (FIGS. 7A and 7B, lanes 6 and 7). Meanwhile, S1 with msp and VSV Gtc was present in the concentrated media and pellet (FIGS. 7A and 7B, lanes 3 and 4) indicating that the S1 with msp at the NH₂ terminus and VSV Gtc at the COOH terminus was secreted and also incorporated into pseudotype VSV particles.

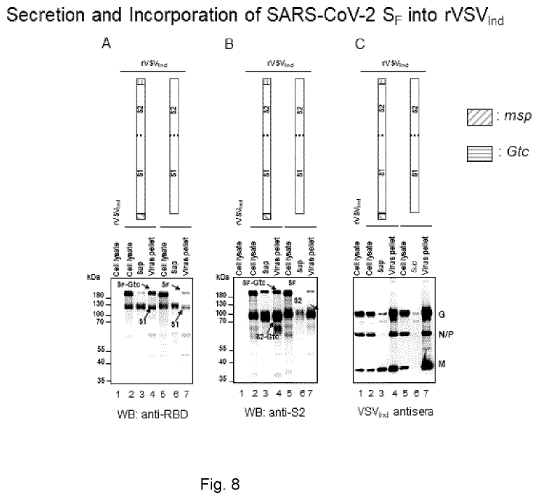

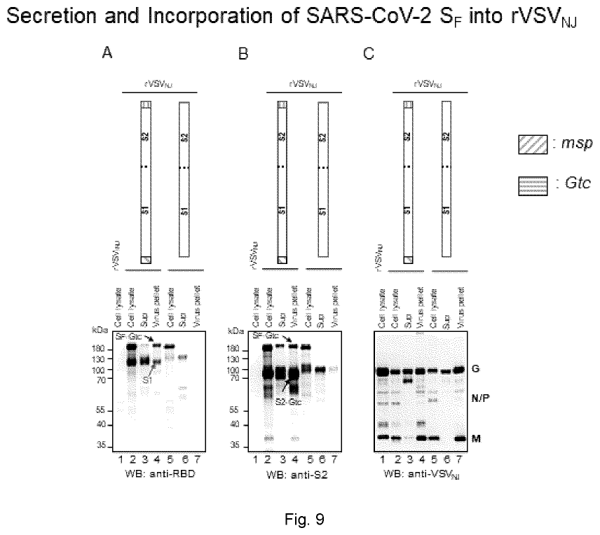

Although full-length SARS-CoV-2 S protein without modifications on both NH₂ and COOH termini could secret from the infected cells and incorporate into VSV particles (FIG. 8, lanes 6 and 7), replacing signal peptides at NH₂ terminus with msp and transmembrane and cytoplasmic tail at the COOH terminus with VSV Gtc increased the secretion and incorporation of full-length S (S_{F}) protein from the cells infected with both serotypes of VSV (FIG. 8A, 8B FIGS. 9A, and 9B lanes 3 and 4). We could detect cleaved S1 and S2 as well as S_{F} proteins in pseudotype VSV particles. The results demonstrated that uncleaved S_{F} and cleaved S2 could incorporate into VSV particles through their transmembrane and cytoplasmic domain. The detection of S1 from the virus particle shows that the cleaved S1 was associated with VSV particles by interacting with the incorporated S2 on the VSV particles.

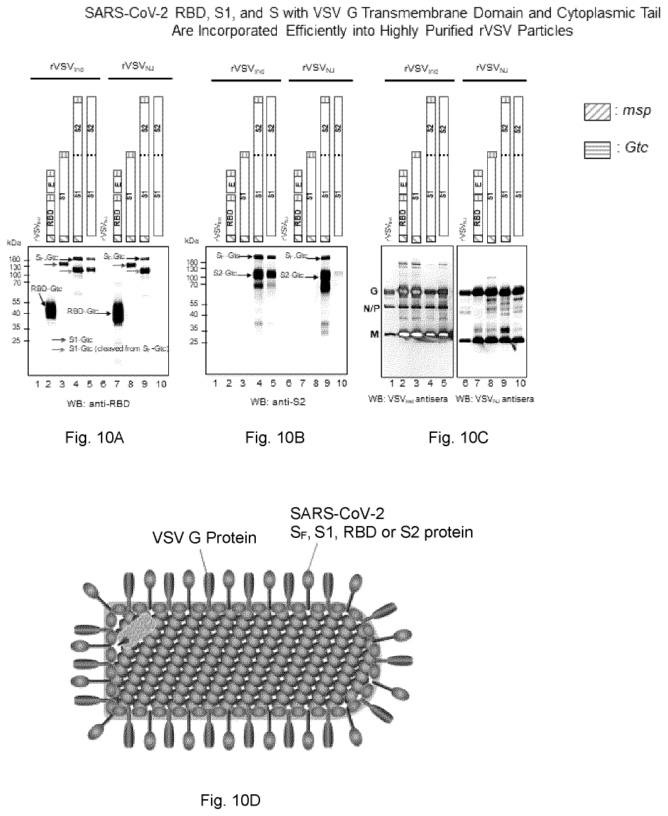

We also examined the presence of SARS-CoV 2 RBD, S1, S2, and S_{F} proteins with the modifications in the purified rVSV-SARS-CoV-2, which were prepared for the vaccination in mice. We purified rVSV-SARS-CoV-2 as well as control viruses without SARS-CoV-2 genes by anion-exchange chromatography using the Mustang Q XT5 membrane capsule (Pall, XTSMSTGQPM6) to examine the immune responses in mice (Table 1). The purified rVSV-SARS-CoV-2 was run on the SDS-PAGE and the presence of RBD, S1, S2, and S_{F} was determined by Western blot analysis. We could detect good amounts of RBD, S1, S_{F} (FIG. 10A, lanes 2, 3, 4, 5, 7, 8, and 9), and S2 (FIG. 10, lanes 4, 5, and 9) in the purified viruses. The results demonstrated that the purified rVSV-SARS-CoV-2 for the vaccination studies had incorporated RBD, S2, S1, and S_{F}.

### 5. S_{F} Protein with the Melittin Signal Peptide at the Amino Terminus and VSV G Protein Transmembrane and Cytoplasmic Tail Domains at the Carboxyl Terminus Induces the Highest IgG Titers Against SARS-CoV-2 S1 Protein

We examined immune responses against SARS-CoV-2 RBD, S1, and S_{F} with and without modifications at NH₂ and COOH termini. We vaccinated five C57BL/6 mice/vaccination group intramuscularly with two different doses, 5×10⁷ PFU/mouse and 5×10⁸ PFU/mouse (Table 2). The mice were prime immunized with rVSV_{Ind} (GML)-SARS-CoV-2 and boost immunized with rVSV_{NJ} (GMM)-SARS-CoV-2 2 weeks after the prime-immunization. Sera were collected to check the SARS-CoV-2 S protein-specific antibody level at day 13, right before the boost-immunization and day 27, 2 weeks after the boost-immunization. We determined the S1 specific IgG level in the day 13 and day 27 sera samples by indirect enzyme-linked immunosorbent assay (ELISA). A 96 well microplate was coated with 100 μl of S1 protein in 2 μg/ml concentration. Serum was diluted by 5-fold serial dilutions starting from 1/30 dilution. Antibody titer was determined by the serum dilutions, which was higher than the negative control serum.

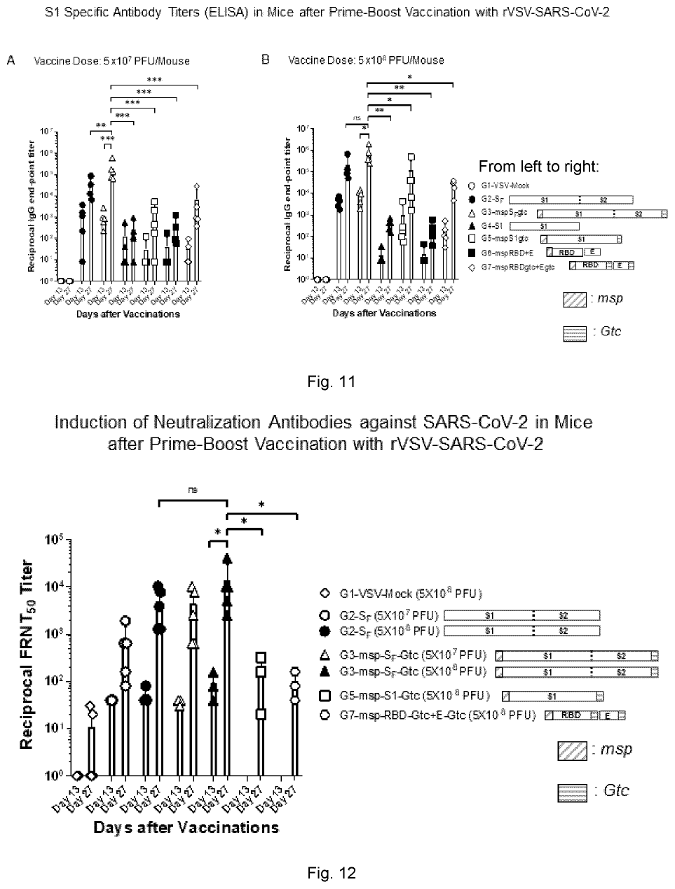

The dose of 5×10⁸ PFU/mouse was better than the dose of 5×10⁷ PFU/mouse to induce S1 specific IgG in mice for all vaccination groups (FIGS. 11A and 11B). For both doses, anti-S1 antibody level was increased after boost-immunization (FIGS. 11A and 11B). The rVSV-msp-S_{F}-Gtc and rVSV-S_{F} induced higher immune responses in both doses than rVSV-msp-S1-Gtc, rVSV-S1, rVSV-msp-RBD+E, and rVSV-msp-RBD-Gtc+E-Gtc (FIG. 11). The vaccination with rVSV-msp-S_{F}-Gtc generated the highest SARS-CoV-2 S1 specific antibody level, although the differences from the antibody level by the rVSV-S_{F} were not statistically significant.

### 6. S_{F} Protein with the Melittin Signal Peptide at the Amino Terminus and VSV G Protein Transmembrane and Cytoplasmic Tail Domains at the Carboxyl Terminus Induce the Highest Neutralizing Antibody Titer Against SARS-CoV-2

For the vaccine to prevent vaccinee from the SARS-CoV-2 infection, the vaccine should induce good quantities of neutralizing antibodies, which will block the entry of the virus to the cells by binding the surface spike protein (S) on the SARS-CoV-2. Therefore, we analyzed the neutralization antibody titers against wild type SARS-CoV-2 in the immune serum by focus reduction neutralization titer (FRNT) assay. Mice were immunized and sera were collected as described in Table 2. We made 2-fold serum dilutions in 25 μl of DMEM w/2% FBS and mixed them with 500 PFU/25 μl/well of live SARS-CoV-2. The serum-virus mixture was incubated at 37° C. for 30 minutes and the 50 μl of the mixture was added to Vero cells in a 96 well microplate. The infected cells were incubated for 4 hrs in the 37° C. CO₂ incubator. After 4 hrs of incubation, the cells were fixed with 4% formaldehyde. A day after the fixation with formaldehyde, the cells were treated with cold 100% methanol for 10 minutes to increase permeability. After blocking with 100 μl of blocking buffer (1% BSA, 0.5% goat serum, 0.1% tween-20 in PBS), the cells were incubated with primary anti-SARS-CoV-2 NP rabbit mAb in 1:3000 dilutions at 37° C. for 1 hr. The cells were washed 3 times with 200 μl of wash buffer (0.1% tween 20 in PBS). A secondary antibody, goat anti-rabbit IgG-HRP in 1:2000 dilutions in the blocking buffer was added to the cells and was incubated at 37° C. for 1 hr. After washing the cells 3 times with wash buffer, 30 μl of tetramethylbenzidine (TMB) was added to the cells and was incubated at room temperature for 30 minutes. The developed foci were count with spot reader (CTL, ImmunoSpot S5). The number of foci, which was developed in the presence and absence of the diluted serum was counted and the highest serum dilutions for the 50% foci number reduction of (FRNT₅₀) were determined.

We checked the FRNT₅₀ for the day 13 and day 27 serum samples from the rVSV-msp-S_{F}-Gtc- and rVSV-S_{F}-vaccinated groups with the doses of 5×10⁷ PFU and 5×10⁸ PFU, which showed the highest level of S1 specific IgG after boost-immunization (Table 3, FIG. 12). We also checked the FRNT₅₀ for the serum samples from the vaccinated mice with 5×10⁸ PFU of rVSV-msp-S1-Gtc and with 5×10⁸ of rVSV-msp-RBD-Gtc+E-Gtc. In both vaccination groups immunized with rVSV-msp-S_{F}-Gtc and rVSV-S_{F}, the neutralizing antibody titer was significantly increased after boost-immunization, especially in groups immunized with the dose of 5×10⁸ PFU (Table 3, FIG. 12). Mice vaccinated with rVSV-msp-S_{F}-Gtc or rVSV-S_{F} produced significantly higher titer of FRNT₅₀ after boost-immunization than the mice vaccinated with rVSV-msp-S1-Gtc or rVSV-msp-RBD-Gtc+E-Gtc (Table 3, FIG. 12). Between the mice groups with rVSV-msp-S_{F}-Gtc or rVSV-S_{F}, the rVSV-msp-S_{F}-Gtc group showed higher titer in both dosage groups than the rVSV-S_{F} group. All mice in the rVSV-msp-S_{F}-Gtc group with the dose of 5×10⁸ PFU had a higher FRNT₅₀ titer than the highest serum dilution, 1/2,560 (Table 3 and FIG. 12). We have carried out another set of experiments focusing on the induction of neutralizing antibodies with rVSV-S_{F} and rVSV-msp-S_{F}-Gtc vaccines, since our previous results (Table 3) showed that the full-length spike protein-based vaccines induced high level of neutralizing antibodies. We found 5×10⁸ PFU/dose of the rVSV-msp-S_{F}-Gtc vaccine induced very high (average of 13,824 FRNT₅₀) of neutralizing antibodies (Table 4). The results demonstrated that prime-immunization with rVSV_{Ind}(GML)-msp-S_{F}-Gtc in the dose of 5×10⁸ PFU and boost-immunization with rVSV_{NJ} (GMM)-msp-S_{F}-Gtc in the dose of 5×10⁸ PFU induced the highest neutralization antibody titers in mice.

### 7. SARS-CoV-2 Challenge Experiments

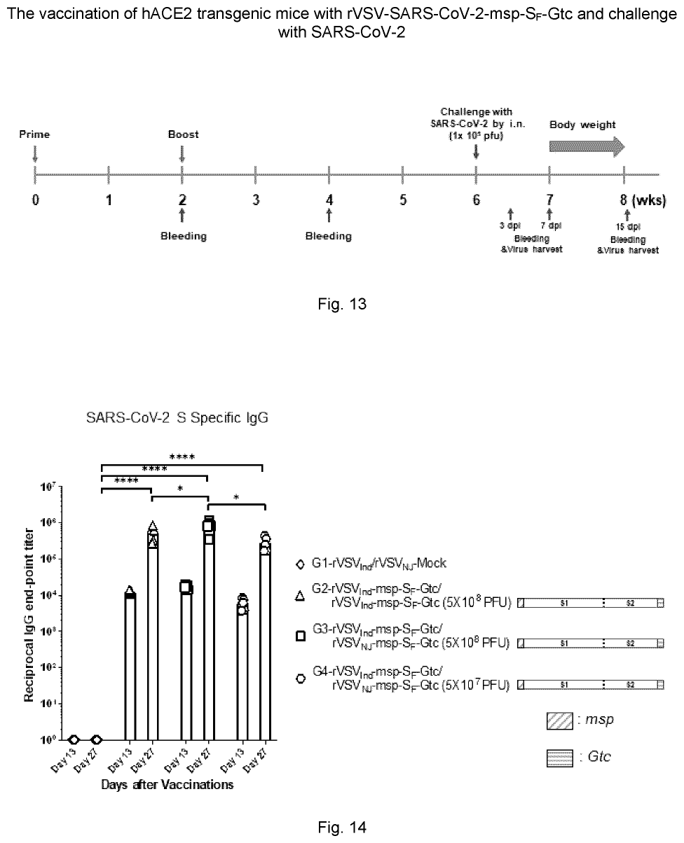

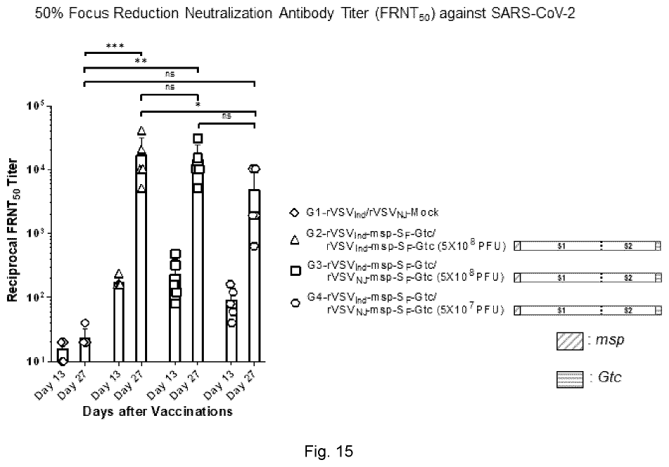

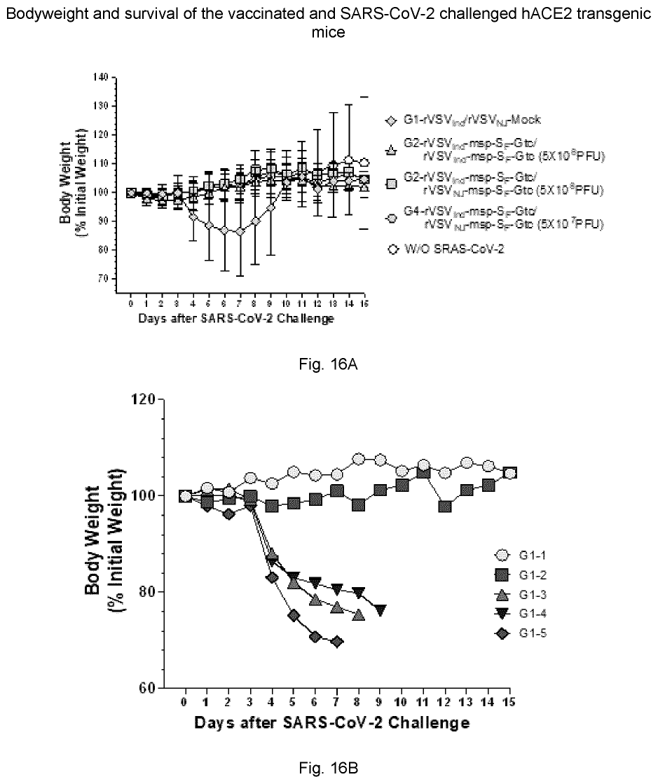

Mice were immunized and sera were collected as described in Table 5 and illustrated in FIG. 13. The groups and individual mice identification are summarized in Table 6. SARS-CoV-2 S specific IgGs were checked at days 13 and 27 for each group. The neutralizing antibody titer was significantly increased after boost-immunization (FIG. 14).

All mice groups produced significantly higher titer of FRNT₅₀ after boost-immunization (FIG. 14). At same dose of 5×10⁸ PFU, there was no significant difference between mice vaccinated with rVSViod-rVSViod prime-boost regime, and those vaccinated with a rVSViod-rVSV_{NJ} prime-boost regime (FIG. 14).

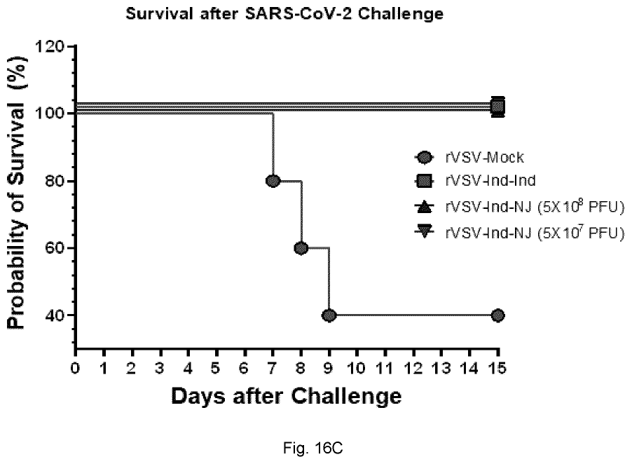

As illustrated in FIG. 16C, mice primed with a vaccine comprising rVSV_{Ind}-msp-S_{f}-Gtc followed by a booster comprising the same rVSV_{Ind} (rVSV-Ind-Ind) and mice primed with a vaccine comprising rVSV_{Ind}-msp-S_{f}-Gtc followed by a booster comprising a rVSV_{NJ}-msp-S_{f}-Gtc (rVSV-Ind-NJ) survived after a SARS-CoV-2 challenge, while 3 out of 5 mice that were mock inoculated (VSV-Mock) did not survive the SARS-CoV-2 challenge.

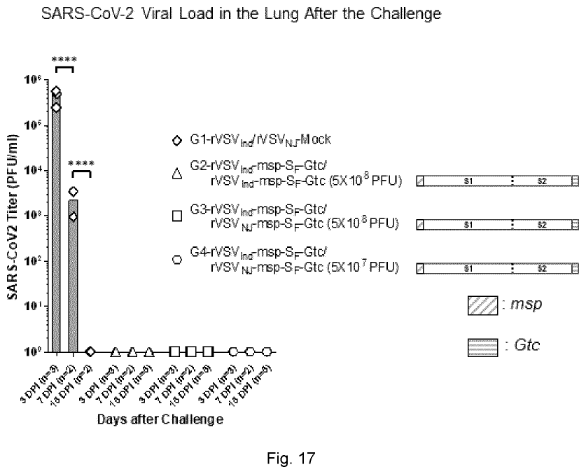

The SARS-CoV-2 viral load in the lung at 3, 7 and 15 days after the SARS-Co-V2 challenge is shown in FIG. 17. Note that only mice that were inoculated with VSV mock showed SARS-CoV-2 viral load in the lung after 3- and 7-days post challenge.

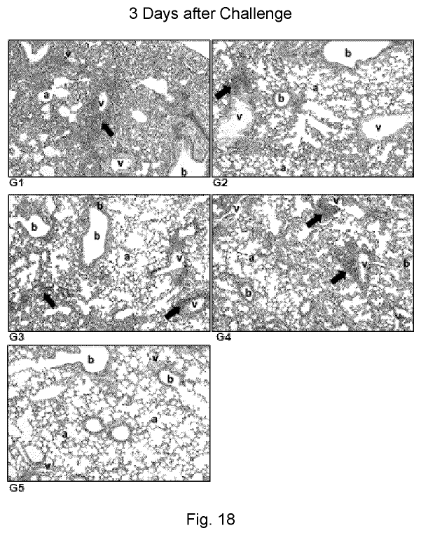

Inflammatory cell infiltration around the blood vessels (thick arrows) were noted in the lungs on day 3 after SARS-CoV-2 infection (see FIG. 18). In the G2-2 group, inflammation was extended into the alveolar areas, with infiltration of mononuclear cells and a few neutrophils in the alveolar spaces (alveolitis).

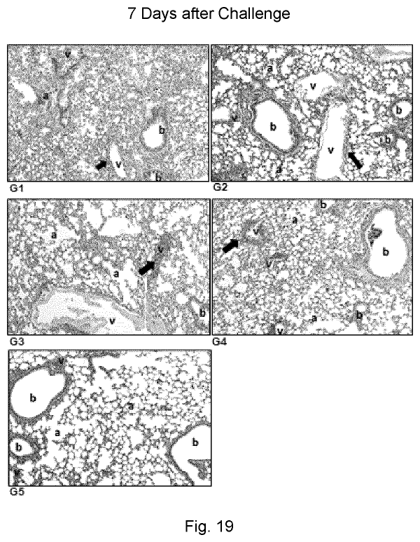

With reference to FIG. 19, on day 7 after SARS-CoV-2 infection, inflammatory cell infiltration was noted around the blood vessels (thick arrows) in the virus infected groups. In the G2-10, alveolitis was also evident with inflammatory cell infiltration in the alveolar spaces.

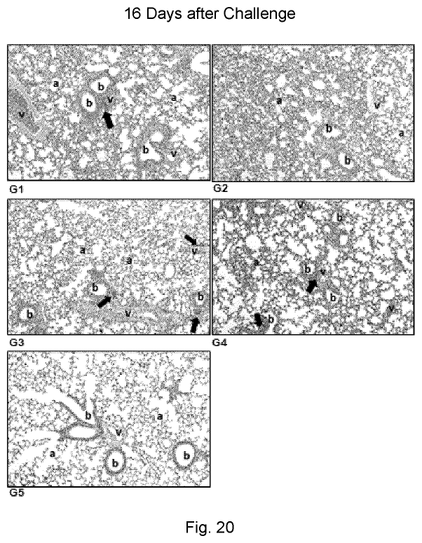

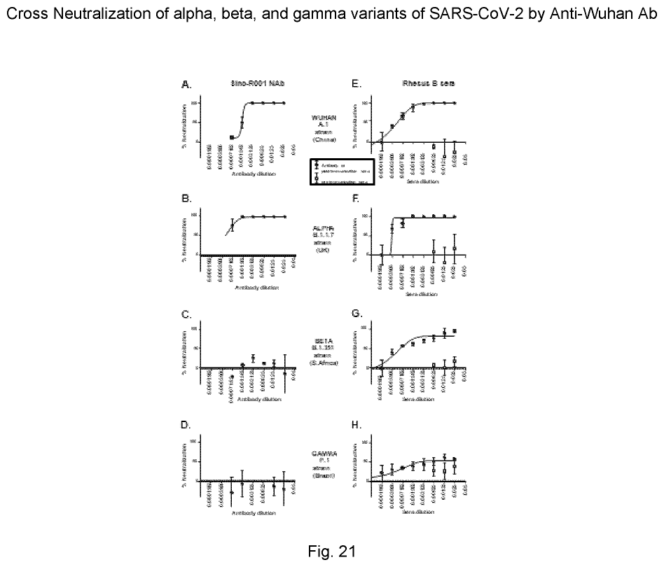

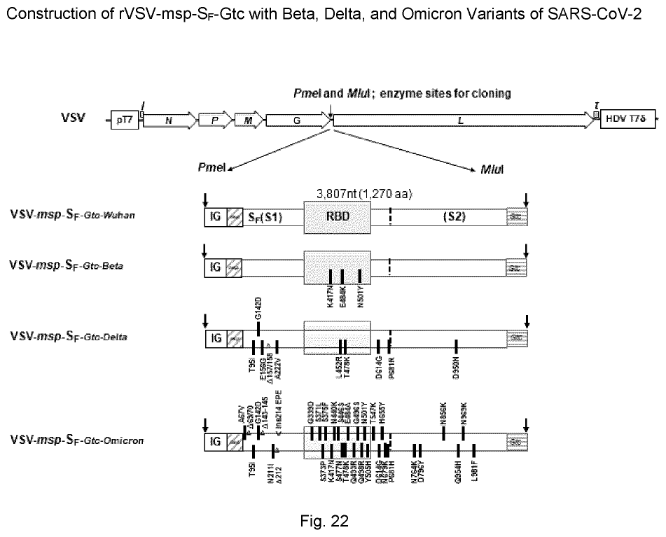

With reference to FIG. 20, on day 16 after SARS-CoV-2 infection, minimal grade of inflammatory cell infiltration around the blood vessels (thick arrows) in the virus infected groups. Inflammatory reaction was considerably attenuated on day 16 in all groups, compared with that on day 3 and day 7.

### REFERENCES

1. Chen N, Zhou M, Dong X, Qu J, Gong F, Han Y, Qiu Y, Wang J, Liu Y, Wei Y, Xia J, Yu T, Zhang X, Zhang L. 2020. Epidemiological and clinical characteristics of 99 cases of 2019 novel coronavirus pneumonia in Wuhan, China: a descriptive study. Lancet 395:507-513.
2. Lu R, Zhao X, Li J, Niu P, Yang B, Wu H, Wang W, Song H, Huang B, Zhu N, Bi Y, Ma X, Zhan F, Wang L, Hu T, Zhou H, Hu Z, Zhou W, Zhao L, Chen J, Meng Y, Wang J, Lin Y, Yuan J, Xie Z, Ma J, Liu W J, Wang D, Xu W, Holmes E C, Gao G F, Wu G, Chen W, Shi W, Tan W. 2020. Genomic characterisation and epidemiology of 2019 novel coronavirus: implications for virus origins and receptor binding. Lancet 395:565-574.
3. Zhou P, Yang X L, Wang X G, Hu B, Zhang L, Zhang W, Si H R, Zhu Y, Li B, Huang C L, Chen H D, Chen J, Luo Y, Guo H, Jiang R D, Liu M Q, Chen Y, Shen X R, Wang X, Zheng X S, Zhao K, Chen Q J, Deng F, Liu L L, Yan B, Zhan F X, Wang Y Y, Xiao G F, Shi Z L. 2020. A pneumonia outbreak associated with a new coronavirus of probable bat origin. Nature 579:270-273.
4. Kim G N, Wu K, Hong J P, Awamleh Z, Kang C Y. 2015. Creation of matrix protein gene variants of two serotypes of vesicular stomatitis virus as prime-boost vaccine vectors. J Virol 89:6338-51.
5. Kim G N, Kang C Y. 2007. Matrix protein of VSV New Jersey serotype containing methionine to arginine substitutions at positions 48 and 51 allows near-normal host cell gene expression. Virology 357:41-53.
6. Gordon D E, Jang G M, Bouhaddou M, Xu J, Obernier K, White K M, O'Meara M J, Rezelj V V, Guo J Z, Swaney D L, Tummino T A, Huttenhain R, Kaake R M, Richards A L, Tutuncuoglu B, Foussard H, Batra J, Haas K, Modak M, Kim M, Haas P, Polacco B J, Braberg H, Fabius J M, Eckhardt M, Soucheray M, Bennett M J, Cakir M, McGregor M J, Li Q, Meyer B, Roesch F, Vallet T, Mac Kain A, Miorin L, Moreno E, Naing Z Z C, Zhou Y, Peng S, Shi Y, Zhang Z, Shen W, Kirby I T, Melnyk J E, Chorba J S, Lou K, Dai S A, Barrio-Hernandez I, Memon D, Hernandez-Armenta C, et al. 2020. A SARS-CoV-2 protein interaction map reveals targets for drug repurposing. Nature 583:459-468.
7. Perrotta F, Matera M G, Cazzola M, Bianco A. 2020. Severe respiratory SARS-CoV2 infection: Does ACE2 receptor matter? Respir Med 168:105996.
8. Walls A C, Park Y J, Tortorici M A, Wall A, McGuire A T, Veesler D. 2020. Structure, Function, and Antigenicity of the SARS-CoV-2 Spike Glycoprotein. Cell 181:281-292 e6.
9. Li Y, Luo L, Thomas D Y, Kang C Y. 1994. Control of expression, glycosylation, and secretion of HIV-1 gp120 by homologous and heterologous signal sequences. Virology 204:266-78.

TABLE 1
The Infectious Titer of the Purified rVSVs-SARS-CoV-2 Vaccines.

| rVSV-CoV-2 | Titer (PFU/ml) |
|---|---|
| rVSV_{Ind}(GML) | 2.0 × 10¹¹ |
| rVSV_{NJ-H}(GMM) | 1.0 × 10¹¹ |
| rVSV_{Ind}(GML)-S_{F} | 1.3 × 10¹¹ |
| rVSV_{NJ-H}(GMM)-S_{F} | 1.1 × 10¹⁰ |
| rVSV_{Ind}(GML)-msp-S_{F}-Gtc | 5.5 × 10¹⁰ |
| rVSV_{NJ-O}(GMM)-msp-S_{F}-Gtc | 4.3 × 10¹⁰ |
| rVSV_{Ind}(GML)-S1 | 1.02 × 10¹¹ |
| rVSV_{NJ-H}(GMM)-S1 | 4.0 × 10¹⁰ |
| rVSV_{Ind}(GML)-msp-S1-Gtc | 1.65 × 10¹¹ |
| rVSV_{NJ-H}(GMM)-msp-S1-Gtc | 2.4 × 10¹¹ |
| rVSV_{Ind}(GML)-msp-RBD + E | 8.25 × 10¹⁰ |
| rVSV_{NJ-H}(GMM)-msp-RBD + E | 1.7 × 10¹⁰ |
| rVSV_{Ind}(GML)-msp-RBD-Gtc + E-Gtc | 1.6 × 10¹¹ |
| rVSV_{NJ-H}(GMM)-msp-RBD-Gtc + E-Gtc | 1.25 × 10¹¹ |

TABLE 2
Vaccination with rVSV-SARS-CoV-2
in Mice - Vaccination Groups

| | | | | | |
|---|---|---|---|---|---|
| G1 | Ind(GML) | NJ-O(GMM) | | | 10 |
| G2 | Ind(GML)-S_{F} | NJ-O(GMM)-S_{F} | | | 10 |
| G3 | Ind(GML)-msp- | NJ-O(GMM)- | | | 10 |
| | S_{F}-Gtc | msp-S_{F}-Gtc | | | |
| G4 | Ind(GML)-S1 | NJ-H(GMM)-S1 | C57BL/6 | [symbol] M | 10 |
| G5 | Ind(GML)-msp- | NJ-H(GMM)-msp- | | | 10 |
| | S1-Gtc | S1-Gtc | | | |
| G6 | Ind(GML)-msp- | NJ-H(GMM)-msp- | | | 10 |
| | RBD + E | RBD + E | | | |
| G7 | Ind(GML)-msp- | NJ-H(GMM)-msp- | | | 10 |
| | RBD-Gtc + E-Gtc | RBD-Gtc + E-Gtc | | | |

Prime immunization: day 0
Boost immunization: Week 2.
Bleeding: day 13.
Whole blood collection: Day 27. [symbol]
indicates data missing or illegible when filed

TABLE 3
Induction Neutralization Antibodies against SARS-CoV-2 in Mice
after Prime-Boost Vaccination with rVSV-SARS-CoV-2 - 50% Focus
Reduction Neutralization antibody titer (FRNT₅₀) against SARS-CoV-2
FRNT₅₀

| | | | | | | | | | | | rVSV-msp- |
|---|---|---|---|---|---|---|---|---|---|---|---|
| | | | | | | rVSV-msp- | | rVSV-msp- | | rVSV-msp- | RBD-Gtc + |
| | rVSV | rVSV-S_{F} | | rVSV-S_{F} | | S_{F}-Gtc | | S_{F}-Gtc | | S1-Gtc | E-Gtc |
| | (5 × 10⁸ | (5 × 10⁷ | | (5 × 10⁸ | | (5 × 10⁷ | | (5 × 10⁸ | | (5 × 10⁸ | (5 × 10₈ |
| | PFU) | PFU) | | PFU) | | PFU) | | PFU) | | PFU) | PFU) |
| Vaccines | Serum Samples | | | | | | | | | | |
| (Dosage) | D 27 | D 13 | | | | | | | | | |
| Animal # | (Pooled) | (Pooled) | D 27 | D 13 | D 27 | D 13 | D 27 | D 13 | D 27 | D 27 | D27 |
| 1 | <20 | 40 | 320 | 160 | ≥2560 | 40 | ≥2560 | 80 | ≥2560 | 80 | 40 |
| 2 | <20 | 40 | 320 | 160 | ≥2560 | 40 | ≥2560 | 80 | ≥2560 | 80 | 40 |
| 3 | <20 | 40 | 160 | 80 | ≥2560 | 40 | ≥2560 | 320 | ≥2560 | <20 | 160 |
| 4 | <20 | 40 | 160 | 80 | ≥2560 | 40 | ≥2560 | 320 | ≥2560 | <20 | 160 |
| 5 | | | ≥2560 | 80 | 640 | 40 | ≥2560 | 160 | ≥2560 | 160 | 20 |
| | | | ≥2560 | 80 | 640 | 40 | ≥2560 | 160 | ≥2560 | 80 | 20 |
| | | | ≥2560 | 160 | 640 | 40 | ≥2560 | 80 | ≥2560 | 40 | 40 |
| | | | ≥2560 | 80 | 640 | 20 | ≥2560 | 80 | ≥2560 | 40 | 40 |
| | | | 160 | 40 | ≥2560 | 40 | 1280 | 160 | ≥2560 | 160 | 640 |
| | | | 320 | 40 | ≥2560 | 40 | 640 | 160 | ≥2560 | 160 | 640 |

TABLE 4
Induction Neutralization Antibodies against SARS-CoV-2 in Mice after Prime-
Boost Vaccination with rVSV-SARS-CoV-2 - 50% Focus Reduction Neutralization antibody
titer (FRNT₅₀) against SARS-CoV-2
FRNT₅₀

| | | | | rVSV-msp- | rVSV-msp- | rVSV-msp- | rVSV-msp-RBD- |
|---|---|---|---|---|---|---|---|
| | rVSV | rVSV-S_{F} | rVSV-S_{F} | S_{F}-Gtc | S_{F}-Gtc | S1-Gtc | Gtc + E-Gtc |
| | (5 × 10⁸ | (5 × 10⁷ | (5 × 10⁸ | (5 × 10⁷ | (5 × 10⁸ | (5 × 10⁸ | (5 × 10₈ |
| | PFU) | PFU) | PFU) | PFU) | PFU) | PFU) | PFU) |
| | Serum Samples | | | | | | |
| | D 27 | D 27 | D 27 | D 27 | D 27 | D 27 | D27 |
| Vaccines | (Sera from 5 | (Average of | (Average of | (Sera from 5 | (Average of | (Sera from 5 | (Sera from 5 |
| (Dosage) | mice are | duplicated | duplicated | mice are | duplicated | mice are | mice are |
| Animal# | pooled) | analyses) | analyses) | pooled) | analyses) | pooled) | pooled) |
| 1 | 25 | 640 | 1280 | 10,240 | 10,240 | 120 | 100 |
| 2 | | 1920 | 1280 | 7,680 | 2,560 | | |
| 3 | | 80 | 1,0240 | 2,560 | 10,240 | | |
| 4 | | 640 | 3,840 | 640 | 5,120 | | |
| 5 | | 160 | 7,680 | 640 | 40,960 | | |
| Average | 25 | 688 | 4,864 | 4,352 | 13,824 | 120 | 100 |

TABLE 5

| | | | | | | Number of mice | |
|---|---|---|---|---|---|---|---|
| | | | | | | Serum | |
| | | | | | | and virus | Body |
| | | | | | | collection | Weight |
| | Prime | Boost | Dose | Vaccination | Challenge | after | and |
| Groups | vaccination | vaccination | (PFU) | Route | Route | challenge | Survival |
| G1 | rVSV_{Ind} | rVSV_{NJ} | 5 × 10⁸ | IM | IN | 5 | 5 |
| | | | | | (SARS- | | |
| | | | | | CoV-2) | | |
| G2 | rVSV_{Ind}- | rVSV_{Ind}- | 5 × 10⁸ | IM | IN | 5 | 5 |
| | msp-S_{F}-Gtc | msp-S_{F}-Gtc | | | (SARS- | | |
| | | | | | CoV-2) | | |
| G3 | rVSV_{Ind}- | rVSV_{NJ}- | 5 × 10⁸ | IM | IN | 5 | 5 |
| | msp-S_{F}-Gtc | msp-S_{F}-Gtc | | | (SARS- | | |
| | | | | | CoV-2) | | |
| G4 | rVSV_{Ind}- | rVSV_{NJ}- | 5 × 10⁷ | IM | IN | 5 | 5 |
| | msp-S_{F}-Gtc | msp-S_{F}-Gtc | | | (SARS- | | |
| | | | | | CoV-2) | | |
| G5 | PBS | PBS | | IM | IN (PBS) | 5 | 5 |

TABLE 6

| Day | | Group | Virus & Treatment | Individual ID | No. of slides |
|---|---|---|---|---|---|
| 3 | dpi | G1 | PBS (No infection) | G1-1, G1-2, G1-3 | 3 |
| | | G2 | rVSV_{IND}-Mock/rVSV_{NJ}-Mock 5 × 10⁸ PFU (SARS-CoV-2 infection) | G2-1, G2-2, G2-3 | 3 |
| | | G3 | SCOV-02/SCOV-08 5 × 10⁸ PFU (SARS-CoV-2 infection) | G3-1, G3-2, G3-3 | 3 |
| | | G4 | SCOV-02/SCOV-02 5 × 10⁸ PFU (SARS-CoV-2 infection) | G4-1, G4-2, G4-3 | 3 |
| | | G5 | SCOV-02/SCOV-08 5 × 10⁷ PFU (SARS-CoV-2 infection) | G5-1, G5-2, G5-3 | 3 |
| 7 | dpi | G1 | PBS (No infection) | G1-4, G1-5 | 2 |
| | | G2 | rVSV_{IND}-Mock/rVSV_{NJ}-Mock 5 × 10⁸ PFU (SARS-CoV-2 infection) | G2-8, G2-10 | 2 |
| | | G3 | SCOV-02/SCOV-08 5 × 10⁸ PFU (SARS-CoV-2 infection) | G3-4, G3-5 | 2 |
| | | G4 | SCOV-02/SCOV-02 5 × 10⁸ PFU (SARS-CoV-2 infection) | G4-4, G4-5 | 2 |
| | | G5 | SCOV-02/SCOV-08 5 × 10⁷ PFU (SARS-CoV-2 infection) | G5-4, G5-5 | 2 |
| 16 | dpi | G1 | PBS (No infection) | G1-6~G1-10 | 5 |
| | | G2 | rVSV_{IND}-Mock/rVSV_{NJ}-Mock 5 × 10⁸ PFU (SARS-CoV-2 infection) | G2-5, G2-6 | 2 |
| | | G3 | SCOV-02/SCOV-08 5 × 10⁸ PFU (SARS-CoV-2 infection) | G3-6~G3-10 | 5 |
| | | G4 | SCOV-02/SCOV-02 5 × 10⁸ PFU (SARS-CoV-2 infection) | G4-6~G4-10 | 5 |
| | | G5 | SCOV-02/SCOV-08 5 × 10⁷ PFU (SARS-CoV-2 infection) | G5-6~G5-10 | 5 |
| | | | | Total | 47 |

TABLE 7
Neucleotide Sequence Comparison between M Genes of VSV Indiana serotype,
Wild Type (SEQ ID NO: 1) and a Mutant G21E/L111A/M51R (SEQ ID NO 2)

| | |
|---|---|
| | 1 50 |
| SEQ ID NO: 1: | ATGAGTTCCT TAAAGAAGAT TCTCGGTCTG AAGGGGAAAG GTAAGAAATC |
| SEQ ID NO: 2: | ATGAGTTCCT TAAAGAAGAT TCTCGGTCTG AAGGGGAAAG GTAAGAAATC |
| | 51 100 |
| SEQ ID NO: 1: | TAAGAAATTA GGGATCGCAC CACCCCCTTA TGAAGAGGAC ACTAACATGG |
| SEQ ID NO: 2: | TAAGAAATTA GAAATCGCAC CACCCCCTTA TGAAGAGGAC ACTAACATGG |
| | 101 150 |
| SEQ ID NO: 1: | AGTATGCTCC GAGCGCTCCA ATTGACAAAT CCTATTTTGG AGTTGACGAG |
| SEQ ID NO: 2: | AGTATGCTCC GAGCGCTCCA ATTGACAAAT CCTATTTTGG AGTTGACGAG |
| | 151 200 |
| SEQ ID NO: 1: | ATGGACACTC ATGATCCGCA TCAATTAAGA TATGAGAAAT TCTTCTTTAC |
| SEQ ID NO: 2: | CGAGACACTC ATGATCCGCA TCAATTAAGA TATGAGAAAT TCTTCTTTAC |
| | 201 250 |
| SEQ ID NO: 1: | AGTGAAAATG ACGGTTAGAT CTAATCGTCC GTTCAGAACA TACTCAGATG |
| SEQ ID NO: 2: | AGTGAAAATG ACGGTTAGAT CTAATCGTCC GTTCAGAACA TACTCAGATG |
| | 251 300 |
| SEQ ID NO: 1: | TGGCAGCCGC TGTATCCCAT TGGGATCACA TGTACATCGG AATGGCAGGG |
| SEQ ID NO: 2: | TGGCAGCCGC TGTATCCCAT TGGGATCACA TGTACATCGG AATGGCAGGG |
| | 301 350 |
| SEQ ID NO: 1: | AAACGTCCCT TCTACAAGAT CTTGGCTTTT TTGGGTTCTT CTAATCTAAA |
| SEQ ID NO: 2: | AAACGTCCCT TCTACAAGAT CTTGGCTTTT GCAGGTTCTT CTAATCTAAA |
| | 351 400 |
| SEQ ID NO: 1: | GGCCACTCCA GCGGTATTGG CAGATCAAGG TCAACCAGAG TATCACGCTC |
| SEQ ID NO: 2: | GGCCACTCCA GCGGTATTGG CAGATCAAGG TCAACCAGAG TATCACGCTC |
| | 401 450 |
| SEQ ID NO: 1: | ACTGTGAAGG CAGGGCTTAT TTGCCACACA GAATGGGGAA GACCCCTCCC |
| SEQ ID NO: 2: | ACTGTGAAGG CAGGGCTTAT TTGCCACACA GAATGGGGAA GACCCCTCCC |
| | 451 500 |
| SEQ ID NO: 1: | ATGCTCAATG TACCAGAGCA CTTCAGAAGA CCATTCAATA TAGGTCTTTA |
| SEQ ID NO: 2: | ATGCTCAATG TACCAGAGCA CTTCAGAAGA CCATTCAATA TAGGTCTTTA |
| | 501 550 |
| SEQ ID NO: 1: | CAAGGGAACG GTTGAGCTCA CAATGACCAT CTACGATGAT GAGTCACTGG |
| SEQ ID NO: 2: | CAAGGGAACG GTTGAGCTCA CAATGACCAT CTACGATGAT GAGTCACTGG |
| | 551 600 |
| SEQ ID NO: 1: | AAGCAGCTCC TATGATCTGG GATCATTTCA ATTCTTCCAA ATTTTCTGAT |
| SEQ ID NO: 2: | AAGCAGCTCC TATGATCTGG GATCATTTCA ATTCTTCCAA ATTTTCTGAT |
| | 601 650 |
| SEQ ID NO: 1: | TTCAGAGATA AGGCCTTAAT GTTTGGCCTG ATTGTCGAGA AAAAGGCATC |
| SEQ ID NO: 2: | TTCAGAGATA AGGCCTTAAT GTTTGGCCTG ATTGTCGAGA AAAAGGCATC |
| | 651 700 |
| SEQ ID NO: 1: | TGGAGCTTGG GTCCTGGATT CTGTCAGCCA CTTCAAATGA |
| SEQ ID NO: 2: | TGGAGCTTGG GTCCTGGATT CTGTCAGCCA CTTCAAATGA |

TABLE 8
Amino Acid Sequence Comparison between M Proteins of VSV Indiana serotype
Wild Type (SEQ ID NO: 3) and a Mutant G21E/L111A/M51R (SEQ ID NO: 4)

| | |
|---|---|
| | 1 21 50 |
| SEQ ID NO: 3: | MSSLKKILGL KGKGKKSKKL GIAPPPYEED TNMEYAPSAP IDKSYFGVDE |
| SEQ ID NO: 4: | MSSLKKILGL KGKGKKSKKL EIAPPPYEED TNMEYAPSAP IDKSYFGVDE |
| | 51 100 |
| SEQ ID NO: 3: | MDTHDPHQLR YEKFFFTVKM TVRSNRPFRT YSDVAAAVSH WDHMYIGMAG |
| SEQ ID NO: 4: | RDTHDPHQLR YEKFFFTVKM TVRSNRPFRT YSDVAAAVSH WDHMYIGMAG |
| | 101 111 150 |
| SEQ ID NO: 3: | KRPFYKILAF LGSSNLKATP AVLADQGQPE YHAHCEGRAY LPHRMGKTPP |
| SEQ ID NO: 4: | KRPFYKILAF AGSSNLKATP AVLADQGQPE YHAHCEGRAY LPHRMGKTPP |
| | 151 200 |
| SEQ ID NO: 3: | MLNVPEHERR PFNIGLYKGT VELTMTIYDD ESLEAAPMIW DHENSSKFSD |
| SEQ ID NO: 4: | MLNVPEHERR PFNIGLYKGT VELTMTIYDD ESLEAAPMIW DHENSSKFSD |
| | 201 229 |
| SEQ ID NO: 3: | FRDKALMFGL IVEKKASGAW VLDSVSHFK |
| SEQ ID NO: 4: | FRDKALMFGL IVEKKASGAW VLDSVSHFK |

TABLE 9
Nucleotide Sequence Comparison between M Genes of VSV New Jersey serotype
Wild Type (SEQ ID NO: 5) and Mutants, G22E/M48R/M51R (SEQ ID NO: 6) and
G22E/L110A/M48R/M51R (SEQ ID NO: 7)

| | |
|---|---|
| | 1 50 |
| SEQ ID NO: 5: | ATGAGTTCCT TCAAAAAGAT TCTGGGATTT TCTTCAAAAA GTCACAAGAA |
| SEQ ID NO: 6: | ATGAGTTCCT TCAAAAAGAT TCTGGGATTT TCTTCAAAAA GTCACAAGAA |
| ID NO: 7: | ATGAGTTCCT TCAAAAAGAT TCTGGGATTT TCTTCAAAAA GTCACAAGAA |
| | 51 100 |
| SEQ ID NO: 5: | ATCAAAGAAA CTAGGCTTGC CACCTCCTTA TGAGGAATCA AGTCCTATGG |
| SEQ ID NO: 6: | ATCAAAGAAA CTAGAATTGC CACCTCCTTA TGAGGAATCA AGTCCTATGG |
| SEQ ID NO: 7: | ATCAAAGAAA CTAGAATTGC CACCTCCTTA TGAGGAATCA AGTCCTATGG |
| | 101 150 |
| SEQ ID NO: 5: | AGATTCAACC ATCTGCCCCA TTATCAAATG ACTTCTTCGG AATGGAGGAT |
| SEQ ID NO: 6: | AGATTCAACC ATCTGCCCCA TTATCAAATG ACTTCTTCGG ACGAGAGGAT |
| SEQ ID NO: 7: | AGATTCAACC ATCTGCCCCA TTATCAAATG ACTTCTTCGG ACGAGAGGAT |
| | 151 200 |
| SEQ ID NO: 5: | ATGGATTTAT ATGATAAGGA CTCCTTGAGA TATGAGAAGT TCCGCTTTAT |
| SEQ ID NO: 6: | CGAGATTTAT ATGATAAGGA CTCCTTGAGA TATGAGAAGT TCCGCTTTAT |
| SEQ ID NO: 7: | CGAGATTTAT ATGATAAGGA CTCCTTGAGA TATGAGAAGT TCCGCTTTAT |
| | 201 250 |
| SEQ ID NO: 5: | GTTGAAGATG ACTGTTAGAG CTAACAAGCC CTTCAGATCG TATGATGATG |
| SEQ ID NO: 6: | GTTGAAGATG ACTGTTAGAG CTAACAAGCC CTTCAGATCG TATGATGATG |
| SEQ ID NO: 7: | GTTGAAGATG ACTGTTAGAG CTAACAAGCC CTTCAGATCG TATGATGATG |
| | 251 300 |
| SEQ ID NO: 5: | TCACCGCAGC GGTATCACAA TGGGATAATT CATACATTGG AATGGTTGGA |
| SEQ ID NO: 6: | TCACCGCAGC GGTATCACAA TGGGATAATT CATACATTGG AATGGTTGGA |
| SEQ ID NO: 7: | TCACCGCAGC GGTATCACAA TGGGATAATT CATACATTGG AATGGTTGGA |
| | 301 350 |
| SEQ ID NO: 5: | AAGCGTCCTT TCTACAAGAT AATTGCTCTG ATTGGCTCCA GTCATCTGCA |
| SEQ ID NO: 6: | AAGCGTCCTT TCTACAAGAT AATTGCTCTG ATTGGCTCCA GTCATCTGCA |
| SEQ ID NO: 7: | AAGCGTCCTT TCTACAAGAT AATTGCTGCA ATTGGCTCCA GTCATCTGCA |
| | 351 400 |
| SEQ ID NO: 5: | AGCAACTCCA GCTGTGTTGG CAGACTTAAA TCAACCAGAG TATTATGCCA |
| SEQ ID NO: 6: | AGCAACTCCA GCTGTGTTGG CAGACTTAAA TCAACCAGAG TATTATGCCA |
| SEQ ID NO: 7: | AGCAACTCCA GCTGTGTTGG CAGACTTAAA TCAACCAGAG TATTATGCCA |
| | 401 450 |
| SEQ ID NO: 5: | CACTAACAGG TCGTTGTTTT CTTCCTCACC GACTCGGATT GATCCCACCG |
| SEQ ID NO: 6: | CACTAACAGG TCGTTGTTTT CTTCCTCACC GACTCGGATT GATCCCACCG |
| SEQ ID NO: 7: | CACTAACAGG TCGTTGTTTT CTTCCTCACC GACTCGGATT GATCCCACCG |
| | 451 500 |
| SEQ ID NO: 5: | ATGTTTAATG TGTCCGAAAC TTTCAGAAAA CCATTCAATA TTGGGATATA |
| SEQ ID NO: 6: | ATGTTTAATG TGTCCGAAAC TTTCAGAAAA CCATTCAATA TTGGGATATA |
| SEQ ID NO: 7: | ATGTTTAATG TGTCCGAAAC TTTCAGAAAA CCATTCAATA TTGGGATATA |
| | 501 550 |
| SEQ ID NO: 5: | CAAAGGGACT CTCGACTTCA CCTTTACAGT TTCAGATGAT GAGTCTAATG |
| SEQ ID NO: 6: | CAAAGGGACT CTCGACTTCA CCTTTACAGT TTCAGATGAT GAGTCTAATG |
| SEQ ID NO: 7: | CAAAGGGACT CTCGACTTCA CCTTTACAGT TTCAGATGAT GAGTCTAATG |
| | 551 600 |
| SEQ ID NO: 5: | AAAAAGTCCC TCATGTTTGG GAATACATGA ACCCAAAATA TCAATCTCAG |
| SEQ ID NO: 6: | AAAAAGTCCC TCATGTTTGG GAATACATGA ACCCAAAATA TCAATCTCAG |
| SEQ ID NO: 7: | AAAAAGTCCC TCATGTTTGG GAATACATGA ACCCAAAATA TCAATCTCAG |
| | 601 650 |
| SEQ ID NO: 5: | ATCCAAAAAG AAGGGCTTAA ATTCGGATTG ATTTTAAGCA AGAAAGCAAC |
| SEQ ID NO: 6: | ATCCAAAAAG AAGGGCTTAA ATTCGGATTG ATTTTAAGCA AGAAAGCAAC |
| SEQ ID NO: 7: | ATCCAAAAAG AAGGGCTTAA ATTCGGATTG ATTTTAAGCA AGAAAGCAAC |
| | 651 700 |
| SEQ ID NO: 5: | GGGAACTTGG GTGTTAGACC AATTGAGTCC GTTTAA |
| SEQ ID NO: 6: | GGGAACTTGG GTGTTAGACC AATTGAGTCC GTTTAA |
| SEQ ID NO: 7: | GGGAACTTGG GTGTTAGACC AATTGAGTCC GTTTAA |

TABLE 10
Amino Acid Sequence Comparison between M Proteins of VSV New Jersey
serotype Wild Type (SEQ ID NO: 8) and Mutants, G22E/M48R/M51R (SEQ ID NO: 9)
and G22E/L110A/M48R/M51R (SEQ ID NO: 10)

| | |
|---|---|
| | 1 22 48 50 |
| SEQ ID NO: 8: | MSSFKKILGF SSKSHKKSKK LGLPPPYEES SPMEIQPSAP LSNDEFGMED |
| SEQ ID NO: 9: | MSSFKKILGF SSKSHKKSKK LELPPPYEES SPMEIQPSAP LSNDFFGRED |
| SEQ ID NO: 10: | MSSFKKILGF SSKSHKKSKK LELPPPYEES SPMEIQPSAP LSNDFFGRED |
| | 51 100 |
| SEQ ID NO: 8: | MDLYDKDSLR YEKFREMLKM TVRANKPERS YDDVTAAVSQ WDNSYIGMVG |
| SEQ ID NO: 9: | RDLYDKDSLR YEKFREMLKM TVRANKPERS YDDVTAAVSQ WDNSYIGMVG |
| SEQ ID NO: 10: | RDLYDKDSLR YEKFREMLKM TVRANKPERS YDDVTAAVSQ WDNSYIGMVG |
| | 101 110 150 |
| SEQ ID NO: 8 | KRPFYKIIAL IGSSHLQATP AVLADLNQPE YYATLTGRCF LPHRLGLIPP |
| SEQ ID NO: 9 | KRPFYKIIAL IGSSHLQATP AVLADLNQPE YYATLTGRCF LPHRLGLIPP |
| SEQ ID NO: 10: | KRPFYKIIAA IGSSHLQATP AVLADLNQPE YYATLTGRCF LPHRLGLIPP |
| | 151 200 |
| SEQ ID NO: 8: | MFNVSETFRK PFNIGIYKGT LDFTFTVSDD ESNEKVPHVW EYMNPKYQSQ |
| SEQ ID NO: 9: | MFNVSETFRK PFNIGIYKGT LDFTFTVSDD ESNEKVPHVW EYMNPKYQSQ |
| SEQ ID NO: 10: | MFNVSETFRK PENIGIYKGT LDFTFTVSDD ESNEKVPHVW EYMNPKYQSQ |
| | 201 250 |
| SEQ ID NO: 8 | IQKEGLKFGL ILSKKATGTW VLDQLSPEK |
| SEQ ID NO: 9 | IQKEGLKFGL ILSKKATGTW VLDQLSPEK |
| SEQ ID NO: 10: | IQKEGLKFGL ILSKKATGTW VLDQLSPEK |

Honeybee melittin signal peptide (msp; SEQ ID NO: 11):
MKFLVNVALVEMVVYISYIYA
VSV G protein transmembrane domain and cytoplasmic tai (Gtc; SEQ ID NO: 12):
SSIASFFFIIGLIIGLFL VLRVGIYLCIKLKHTKKRQIYTDIEMNRLGK
VSV intergenic junctions (SEQ ID NO: 13):
catatgaaaaaaactaacagatatc

1: A recombinant vesicular stomatitis virus (rVSV) carrying a vesicular stomatitis virus glycoprotein (G) gene and a gene that encodes for a full length spike (S_{F}) protein of SARS-CoV 2.

2-5. (canceled)

6: The rVSV of claim 1, wherein the gene encodes for the S_{F} protein having a signal peptide sequence of wild type S_{F} protein replaced with a honeybee melittin peptide (msp) at the NH₂-terminus of the S_{F} protein and a VSV G protein transmembrane domain and cytoplasmic tail (Gtc) at the C-terminus of the S_{F} protein (rVSV-msp-S_{F}-Gtc).

7-9. (canceled)

10: The rVSV of claim 6, wherein the Gtc is a VSV_{Ind} Gtc or a VSV_{NJ} Gtc.

11: The rVSV of claim 1, wherein the rVSV is a replication competent rVSV of Indiana (VSV_{Ind}) serotype having a mutant matrix protein (M) gene, or replication competent rVSV of New Jersey (VSV_{NJ}) serotype having a mutant M gene.

12-13. (canceled)

14: The rVSV claim 11, wherein the M gene of the rVSV_{Ind} includes a GML mutation (rVSV_{Ind}-GML), and the M gene of the rVSV_{NJ} includes a GMM mutation (rVSV_{NJ}-GMM) or a GMML mutation (rVSV_{NJ}-GMML).

15-18. (canceled)

19. The rVSV of claim 1, wherein the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

20: A vaccine or immunological composition including a recombinant vesicular stomatitis virus (rVSV) of claim 1.

21-22. (canceled)

23: The vaccine or immunological composition of claim 20, wherein the gene encodes for the S_{F} having a signal peptide sequence of wild type S_{F} protein replaced with a honeybee melittin peptide (msp) at the NH₂-terminus of the S_{F} protein and a VSV G protein transmembrane domain and cytoplasmic tail (Gtc) at the C-terminus of the S_{F} protein (rVSV-msp-S_{F}-Gtc).

24-26. (canceled)

27: A prime boost immunization combination against SARS-CoV-2 including: (a) a prime vaccine or immunogenic composition comprising a replication competent recombinant vesicular stomatitis virus (rVSV) carrying a vesicular stomatitis virus qlycoprotein (G) gene and a gene that encodes for a spike (S) protein of a SARS-CoV-2, and (b) a booster vaccine or immunogenic composition comprising a replication competent rVSV carrying the vesicular stomatitis virus qlycoprotein (G) gene and the gene that encodes for the S protein, wherein the S protein is full length (S_{F}) or partial length.

28-30. (canceled)

31: The prime boost immunization combination against SARS-CoV-2 of claim 27, wherein the one or more genes encode for the S protein, the S protein is the S_{F} protein having a honeybee melittin peptide (msp) at the NH₂-terminus of the S_{F} protein and a VSV G protein transmembrane domain and cytoplasmic tail (Gtc) at the C-terminus of the S_{F} protein (rVSV-msp-S_{F}-Gtc).

32-34. (canceled)

35: The prime boost immunization combination against SARS-CoV-2 of claim 27, wherein the rVSV of the prime vaccine or immunogenic composition and the rVSV of the booster vaccine or immunogenic composition are rVSV of the same serotype.

36-37. (canceled)

38: The prime boost immunization combination against SARS-CoV-2 of claim 27, wherein the rVSV of the prime vaccine or immunogenic composition is Indiana serotype (VSV_{Ind}) and the rVSV of the booster vaccine or immunogenic composition is New Jersey (VSV_{NJ}), or the rVSV of the prime vaccine or immunogenic composition is rVSV_{NJ} and the rVSV of the booster vaccine or immunogenic composition is rVSV_{Ind}, or the rVSV of the prime vaccine or immunogenic composition is rVSV_{Ind} and the rVSV of the booster vaccine or immunogenic composition is rVSV_{NJ}, or the rVSV of the prime vaccine or immunogenic composition and the rVSV of the booster vaccine or immunogenic composition are rVSV_{Ind}, or the rVSV of the prime vaccine or immunogenic composition and the rVSV of the booster vaccine or immunogenic composition are rVSV_{NJ}.

39. (canceled)

40: The prime boost immunization combination against SARS-CoV-2 of claim 27, wherein the rVSV of the prime vaccine and the rVSV of the booster vaccine include a mutant matrix protein (M) gene.

41: The prime boost immunization combination against SARS-CoV-2 of claim 40, wherein when the rVSV is rVSV_{Ind}, the M protein includes a GML mutation (rVSV_{Ind}-GML) and when the rVSV is rVSV_{NJ} the M protein includes a GMM mutation (rVSV_{NJ}-GMM) or a GMML mutation (rVSV_{NJ}-GMML).

42-43. (canceled)

44. The prime boost immunization combination against SARS-CoV-2 of claim 27, wherein the SARS-CoV-2 include wild-type SARS-CoV-2 and variants of SARS-CoV-2 including Alpha, Beta, Delta, Gamma, Epsilon, Eta, Iota, Kappa, 1.617.3, Mu, Omicron, and Zeta variants of SARS-CoV-2 and their respective descendent lineages.

45: A method for inducing an immune response in a mammal against SARS-CoV-2 or preventing an infection caused by SARS-CoV-2, comprising administering to the mammal an effective amount of a vaccine or immunogenic composition of claim 20.

46: A method for inducing an immune response in a mammal against SARS-CoV-2 or preventing an infection caused by SARS-CoV-2, comprising administering to the mammal the prime boost immunization combination of claim 27.

47-78. (canceled)
